(19) United States
(12) Patent Application Publication
(10) Pub. No.: US 2007/0014786 A1
(43) Pub. Date: Jan. 18, 2007

(54) Methods for treating taxol-induced gut disorder

(75) Inventor: David L. Shelton, Oakland, CA (US)

Correspondence Address: MORRISON & FOERSTER LLP, 755 PAGE MILL RD, PALO ALTO, CA, 94304-1018, US

(73) Assignee: RINAT NEUROSCIENCE CORP., Palo Alto, CA (US)

(21) Appl. No.: 10/549,441

(22) PCT Filed: Mar. 22, 2004
(86) PCT No.: PCT/US04/08865

§ 371 Date: Jun. 26, 2006

Related U.S. Application Data
(60) Provisional application No. 60/456,648, filed on Mar. 20, 2003.

(51) Int. Cl. A61K 39/395

(52) U.S. Cl. 424/133.1; 424/146.1

(57) ABSTRACT

This invention relates to the field of taxol-induced gut disorder. More specifically, the invention relates to methods of treating taxol-induced gut disorder comprising administration of agonist anti-trkC antibody for the treatment, prevention, and/or amelioration of a symptom of taxol-induced gut disorder.

### CROSS-REFERENCE TO RELATED APPLICATIONS

This application claims the priority benefit of provisional application U.S. Ser. No. 60/456,648, filed Mar. 20, 2003, the contents of which are incorporated by reference in their entirety.

### STATEMENT REGARDING FEDERALLY SPONSORED RESEARCH OR DEVELOPMENT

Not applicable.

### FIELD OF THE INVENTION

This invention relates to the field of taxol-induced gut disorder. More specifically, the invention relates to methods of treating taxol-induced gut disorder comprising administration of agonist anti-trkC antibody for the treatment, prevention, and/or amelioration of a symptom of taxol-induced gut disorder.

### BACKGROUND OF THE INVENTION

Chemotherapeutic agents, such as taxol and other taxanes, have been successfully used in treating cancer. Approximately 300,000 people in the U.S. alone will undergo chemotherapy treatment each year for cancers of the breast, lung and colon. However, a percentage of patients treated with taxol can experience gastrointestinal symptoms. For example, a small percentage of patients report intestinal obstruction, intestinal perforation, ischemic colitis, and other gut disorders. See “Taxol (paclitaxel) Product Information Sheet and Patient Information”, Bristol-Meyers Squibb, Inc. (available at http://www.taxol.com).

Neurotrophins are a family of small, homodimeric proteins, which play a crucial role in the development and maintenance of the nervous system. Members of the neurotrophin family include nerve growth factor (NGF), brain-derived neurotrophic factor (BDNF), neurotrophin-3 (NT-3), neurotrophin-4/5 (NT4/5), neurotrophin-6 (NT-6), and neurotrophin-7 (NT-7). Neurotrophins, similar to other polypeptide growth factors, affect their target cells through interactions with cell surface receptors. According to current knowledge, two kinds of transmembrane glycoproteins serve as receptors for neurotrophins. Neurotrophin-responsive neurons possess a common low molecular weight (65-80 kDa), low affinity receptor (LNGFR), also termed as p75NTR or p75, which binds NGF, BDNF, NT-3 and NT-4/5 with a KD of 2×10⁻⁹ M; and large molecular weight (130-150 kDa), high-affinity (KD in the 10⁻¹¹ M range) receptors, which are members of the trk family of receptor tyrosine kinases. The identified members of the trk receptor are trkA, trkB, and trkC.

TrkC is widely expressed in the central nervous system, and on a subset of neurons in the peripheral nervous system. For example, it is expressed on sympathetic neurons and on a subset of primary sensory neurons of the DRG, the large fiber sensory neurons of the DRG.

The extracellular domains of full-length native trkA, trkB and trkC receptors have five structural domains that have been defined with reference to homologous or otherwise similar structures identified in various other proteins. The domains have been designated starting at the N-terminus of the amino acid sequence of the mature trk receptors as 1) a first cysteine-rich domain; 2) a leucine-rich domain; 3) a second cysteine-rich domain; 4) a first immunoglobulin-like domain; and 5) a second immunoglobulin-like domain. See, e.g., PCT WO 0198361; Urfer et al. J. Biol. Chem. 273: 5829-5840 (1998).

Neurotrophins are of interest as potential therapeutic agents for a variety of diseases. Using neurotrophins in treatment of diseases has several shortcomings. One significant shortcoming is the lack of specificity. Most neurotrophins cross-react with more than one receptor. For example NT-3, the preferred ligand of the trkC receptor tyrosine kinase, also binds to and activates trkA and trkB (Barbacid, J. Neurobiol. 25:1386-1403 [1994]; Barbarcid, Ann. New York Aced. Sci. 766:442-458 [1995]; Ryden and Ibanez, J. Biol. Chem. 271:5623-5627 [1996], Belliveau et al., J. Cell. Biol. 136:375-388 [1997]; Farinas et al., Neuron 21:325-334 [1998]). As a result, it is difficult to devise therapies that target a specific population of neurons. Another limitation of neurotrophin therapy is that neurotrophins, including NT-3, are known to elicit hyperalgesia (Chaudhry et al., Muscle and Nerve 23:189-192 [2000]). In addition, some neurotrophins such as NT-3 have poor pharmacokinetic and bioavailability properties in rodents, which raise serious questions about their human clinical applications (Haase et al., J Neurol. Sci. 160:S97-S105 [1998], dosages used in Helgren et al., J. Neurosci. 17(1):372-82 [1997]).

U.S. Ser. No. 10/745,890 and PCT/US03/41367 describe methods for treating taxol-induced sensory neuropathy by administering agonist anti-trkC antibody.

There is a great need for new therapeutic treatment for taxol-induced gut disorders.

All references cited herein, including patent applications and publications, are incorporated by reference in their entirety.

### DISCLOSURE OF THE INVENTION

The present invention is based on the discovery that agonist anti-trkC antibodies treat a gut disorder present in individuals treated with taxol. Taxol-induced gut disorder refers to a gut disorder associated with or present in an individual following administration of the agent, taxol, or related taxanes. Thus, the present invention encompasses methods of treating, preventing, delaying the development of a symptom of, increasing the rate of recovery from, and/or palliating taxol-induced gut disorder using agonist anti-trkC antibodies.

Accordingly, in one aspect, the invention provides methods for treating a taxol-induced gut disorder in an individual comprising administering an effective amount of agonist anti-trkC antibody. In another aspect, the invention provides methods of delaying development of a symptom associated with a taxol-induced gut disorder in an individual comprising treatment of that individual with an effective amount of agonist anti-trkC antibody. In another aspect, the invention provides methods of ameliorating a symptom of a taxol-induced gut disorder in an individual comprising administering an effective amount of agonist anti-trkC antibody. In another aspect, the invention provides methods for reversing a preexisting taxol-induced gut disorder and/or increasing the rate of recovery from a preexisting taxol induced gut disorder by treatment with an effective amount of agonist anti-trkC antibody. In another aspect, the treatment of cancer with taxol can also be enhanced as described herein, by administration of taxol in conjunction with an agonist anti-trkC antibody. In some embodiments, the cancer is any one or more of: breast cancer, ovarian cancer, lung cancer, Kaposi's sarcoma, prostate cancer, head and neck cancers, and hematological malignancies.

The individual may be a mammal. In some embodiments, the individual is a human. Any agonist anti-trkC antibody may be used in the methods of the invention. In some embodiments, the agonist anti-trkC antibody binds human trkC. In some embodiments, the agonist anti-trkC antibody specifically binds human trkC. The agonist anti-trkC antibody may also bind human and rodent trkC. The agonist anti-trkC antibody may be a human antibody (such as antibody 6.4.1 (PCT Publication No. WO 01/98361)) or may be a humanized antibody (including humanized monoclonal antibody 2256). In another embodiment, the humanized agonist anti-trkC antibody is antibody A5, as described herein. In still other embodiments, the anti-trkC agonist antibody comprises the amino acid sequence of the heavy chain variable region shown in Table 3 (SEQ ID NO:1) and the amino acid sequence of the light chain variable region shown in Table 4 (SEQ ID NO:2). In other embodiments, the anti-trkC agonist antibody comprises one or more CDR(s) of antibody A5 (such as one, two three, four, five or, in some embodiments, all six CDRs from A5). Identification of CDRs is well within the skill of the art. In some embodiments, the CDRs comprise the Kabat CDR. In other embodiments, the CDRs are the Chothia CDR. In still other embodiments, the CDR comprises both the Kabat and Chothia CDRs. In some embodiments, the antibody comprises a light chain that is encoded by a polynucleotide with a deposit number of ATCC No. PTA-5682. In some embodiments, the antibody comprises a heavy chain that is encoded by a polynucleotide with a deposit number of ATCC No. PTA-5683. In some embodiments, the antibody comprises (a) a light chain that is encoded by a polynucleotide with a deposit number of ATCC No. PTA-5682; and (b) a heavy chain that is encoded by a polynucleotide with a deposit number of ATCC No. PTA-5683. In some embodiments, the antibody comprises one or more CDR(s) from (a) a light chain that is encoded by a polynucleotide with a deposit number of ATCC No. PTA-5682; and/or (b) a heavy chain that is encoded by a polynucleotide with a deposit number of ATCC No. PTA-5683.

The antibody may bind essentially the same trkC epitope as an antibody selected from any one or more of the following: 6.1.2, 6.4.1, 2345, 2349, 2.5.1, 2344, 2248, 2250, 2253, and 2256. See PCT Publication No. WO 01/98361. The antibody may comprise a modified constant region, such as a constant region that is immunologically inert, e.g., does not trigger a complement mediated lysis or does not stimulate antibody-dependent cell mediated cytotoxicity (ADCC). In other embodiments, the constant region is modified as described in Eur. J. Immunol. (1999) 29:2613-2624; PCT Application No. PCT/GB99/01441; and/or UK Patent Application No. 9809951.8.

The antibody may also be an antibody fragment, such as an antibody fragment selected from one or more of the following: Fab, Fab′, F(ab′)₂, Fv fragments, diabodies, single chain antibody molecules and multispecific antibodies formed from antibody fragments, and a single-chain Fv (scFv) molecule. The antibody may also be chimeric, and it may be bispecific.

The agonist anti-trkC antibody can be administered prior to, during or after administration of taxol, and/or can be delivered before initiating course of taxol therapy; during a course of taxol therapy, and/or after cessation of a course of taxol therapy. Administration can be before onset of gut disorder.

Administration of an agonist anti-trkC antibody can be by any suitable method known in the art, including one or more of the following means: intravenously, subcutaneously, via inhalation, intrarterially, intramuscularly, intracardially, intraventricularly, intrathecally, intraspinally, and intraperitoneally. Administration may be systemic (e.g. intravenously) and/or localized. Administration may be acute and/or chronic.

In another aspect, methods of the invention are effected by administration of a polynucleotide (such as DNA) encoding an agonist anti-trkC antibody.

In another aspect, the invention provides compositions and kits comprising an agonist anti-trkC antibody for use in any of the methods of the invention.

The invention also provides any of the compositions and kits described for any use described herein whether in the context of use as medicament and/or use for manufacture of a medicament.

### BRIEF DESCRIPTION OF THE DRAWINGS

FIG. 1 shows that treatment with taxol results in taxol-induced gut disorder in a mouse model, and that treatment with an anti-trkC agonist antibody ameliorates the gut disorder induced by taxol treatment. Mice were treated with mouse monoclonal antibody 2256 (see PCT WO 01/98361; Urfer et al. J. Biol. Chem. (1998) 273:5829-5840)) subcutaneously (delivered under the scruff) at 0 mg/kg, 0.5 mg/kg, 2 mg/kg or 8 mg/kg on days 0 and day 7. Taxol was given intraperitoneally (IP) on days 1, 3, and 5, at a total dose of 300 mg/m2, split evenly into the three doses. Abdominal distension was scored using a 5 point scale as follows: 0-no noticeable abdominal bulge; 1-slight bulge; 2-distinct bulge; 3-fat mouse; 4-definite lumpy abdomen; 5-swollen abdomen. Results are shown as the percentage of animals that scored either 4 or 5.

FIG. 2 shows that treatment with 300, 375 or 450 mg/m2 of taxol resulted in gut distension.

FIG. 3 shows that treatment with anti-trkC agonist antibody (mouse monoclonal antibody 2256 (see PCT WO 01/98361; Urfer et al. J. Biol. Chem. (1998) 273:5829-5840)) ameliorated taxol-induced gut distension in animals treated with 300 or 450 mg/m² of taxol.

FIG. 4 shows that there was a significant increase in the weight of the GI tract from stomach through colon in animals treated with taxol at 450 mg/m2, and this was partially ameliorated by treatment with anti-trkC agonist Mab 2256.

FIG. 5 shows that there was a significant increase in the weight of the small intestine in animals treated with taxol at 450 mg/m2, and this was partially ameliorated by treatment with anti-trkC agonist Mab 2256.

FIG. 6 shows that there was a significant increase in the weight of the caecum plus large intestine in animals treated with taxol at 450 mg/m2, and this was ameliorated by treatment with anti-trkC agonist Mab 2256.

FIG. 7 shows a photograph of exemplary animals that were rated 1-5, respectively.

FIG. 8 shows that treatment with taxol at either 450 mg/m2 or 375 mg/m2 (four weeks or six weeks after treatment) caused a significant decrease in the length of the GI tract, and this was ameliorated by treatment with anti-trkC agonist Mab 2256.

FIG. 9 shows that treatment with taxol at either 450 mg/m2 or 375 mg/m2 (four weeks or six weeks after treatment) caused a significant increase in the raw weight of the caecum plus large intestine, and this was ameliorated by treatment with anti-trkC agonist Mab 2256.

### MODES FOR CARRYING OUT THE INVENTION

The present invention is based on the discovery that agonist anti-trkC antibodies treat gut disorder present in individuals treated with taxol. The present invention encompasses methods and compositions useful for treating, preventing, delaying the development of a symptom of, increasing the rate of recovery from, and/or palliating taxol-induced gut disorder using agonist anti-trkC antibodies.

Accordingly, in one aspect, the invention provides methods for treating a taxol-induced gut disorder in an individual comprising administering an effective amount of agonist anti-trkC antibody. In another aspect, the invention provides methods of delaying development of a symptom associated with a taxol-induced gut disorder in an individual comprising treatment of that individual with an effective amount of agonist anti-trkC antibody. In another aspect, the invention provides methods of ameliorating a symptom of a taxol-induced gut disorder in an individual comprising administering an effective amount of agonist anti-trkC antibody. In another aspect, the invention provides methods for reversing and/or increasing the rate of recovery from a preexisting taxol induced gut disorder by treatment with an effective amount of agonist anti-trkC antibody. In another aspect, the treatment of cancer with taxol can also be enhanced as described herein, by administration of taxol in conjunction with an agonist anti-trkC antibody. In another aspect, the agonist anti-trkC antibody is administered to an individual at risk for developing taxol-induced gut disorder (an asymptomatic individual).

The agonist anti-trkC antibody can be administered prior to, during and/or after administration of taxol. Alternately, the antibody can be administered in conjunction with the taxol. The agonist anti-trkC antibody can be can be administered prior to, during and/or after administration of any other therapeutic modality for the gut disorder. Alternately, the antibody can be administered in conjunction with any other therapeutic modality for the gut disorder.

Administration of an agonist anti-trkC antibody can be by one or more of the following means: intravenously, subcutaneously, via inhalation, intrarterially, intramuscularly, intracardially, intraventricularly, and intraperitoneally. Administration may be systemic (e.g. intravenously), or localized. Administration may be acute or chronic.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

Definitions

An “antibody” (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)2, Fv), single chain (ScFv), mutants thereof, fusion proteins comprising an antibody portion, humanized antibodies, chimeric antibodies, diabodies linear antibodies, single chain antibodies, multispecific antibodies (e.g., bispecific antibodies) and any other modified configuration of the immunoglobulin molecule that comprises an antigen recognition site of the required specificity. An antibody includes an antibody of any class, such as IgG, IgA, or IgM, and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, gamma, epsilon, and mu, respectively. There are also two classes of light chain, designated kappa and lambda. The subunit structures and three-dimensional configurations of different classes of immunoglobulins are well known.

A “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single antigenic site. The term “monoclonal antibody” encompasses not only intact monoclonal antibodies and full-length monoclonal antibodies, but also fragments thereof (such as Fab, Fab′, F(ab′)2, Fv), single chain (ScFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.).

“Humanized” antibodies refer to a molecule having an antigen binding site that is substantially derived from an immunoglobulin from a non-human species and the remaining immunoglobulin structure of the molecule based upon the structure and/or sequence of a human immunoglobulin. The antigen binding site may comprise either complete variable domains fused onto constant domains or only the complementarity determining regions (CDRs) grafted onto appropriate framework regions in the variable domains. Antigen binding sites may be wild type or modified by one or more amino acid substitutions, e.g., modified to resemble human immunoglobulin more closely. Some forms of humanized antibodies preserve all CDR sequences (for example, a humanized mouse antibody which contains all six CDRs from the mouse antibodies). Other forms of humanized antibodies have one or more CDRs (one, two, three, four, five, six) which are altered with respect to the original antibody.

“Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. Typically, in these chimeric antibodies, the variable region of both light and heavy chains mimics the variable regions of antibodies derived from one species of mammals, while the constant portions are homologous to the sequences in antibodies derived from another. One clear advantage to such chimeric forms is that, for example, the variable regions can conveniently be derived from presently known sources using readily available hybridomas or B cells from non human host organisms in combination with constant regions derived from, for example, human cell preparations. While the variable region has the advantage of ease of preparation, and the specificity is not affected by its source, the constant region being human, is less likely to elicit an immune response from a human subject when the antibodies are injected than would the constant region from a non-human source. However, the definition is not limited to this particular example.

An epitope that “specifically binds” or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a trkC epitope is an antibody that binds this trkC epitope with greater affinity, avidity, more readily, and/or with greater duration than it binds to other trkC epitopes or non-trkC epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

An “agonist anti-trkC antibody” (interchangeably termed “anti-trkC agonist antibody”) refers to an antibody that is able to bind to and activate a trkC receptor and/or downstream pathway(s) mediated by the trkC signaling function. For example, the agonist antibody may bind to the extracellular domain of a trkC receptor and thereby cause dimerization of the receptor, resulting in activation of the intracellular catalytic kinase domain. Consequently, this may result in stimulation of growth and/or differentiation of cells expressing the receptor in vitro and/or in vivo. In some embodiments, an agonist anti-trkC antibody binds to trkC and activates a trkC biological activity. In some embodiments, an agonist antibody useful in the methods of the invention recognizes domain V and/or domain IV of trkC. See Urfer et al., J. Biol. Chem. 273: 5829-5840 (1998).

As used herein, “trkC” refers to the trkC receptor polypeptide, a member of the tyrosine kinase superfamily. TrkC encompasses the native trkC receptor of any mammalian species, including but not limited to, human, canine, feline, bovine, equine, primate, and rodent (including mouse and rat). The extracellular domain of full-length native trkC has been defined with reference to homologous or otherwise similar structures identified in various other proteins. The domains have been designated starting at the N-terminus of the mature trkC receptor as: 1) a first cysteine-rich domain extending from amino acid 1 to amino acid 48; 2) a leucine-rich domain extending from amino acid 49 to amino acid 120 3) a second cysteine-rich domain extending from amino acid 121 to amino acid 177; 4) a first immunoglobulin-like domain extending from about amino acid 196 to amino acid 257; and 5) a second immunoglobulin-like domain extending from about amino acid 288 to amino acid 351. See, e.g., PCT WO 0198361. The domain structure of the trkC receptor has also been designated by reference to a crystal structure as follows: domain 1 from amino acid 1 to amino acid 47; domain 2 from amino acid 48 to amino acid 130; domain 3 from amino acid 131 to amino acid 177; domain 4 from amino acid 178 to amino acid 165; and domain 5 from amino acid 166 to amino acid 381 See, e.g., PCT WO 0198361; Urfer et al. J. Biol. Chem. 273: 5829-5840 (1998). Also included are variants of trkC, examples of which include, but are not limited to, variants without a kinase domain (Shelton, et al., J. Neurosci. 15(1):477-491 [1995]), and variants with a modified kinase domain (Shelton, et al., J. Neurosci. 15(1):477-491 [1995]).

“Biological activity”, when used in conjunction with the agonist anti-trkC antibodies of the present invention, generally refers to having the ability to bind and activate the trkC receptor tyrosine kinase and/or a downstream pathway mediated by the trkC signaling function. As used herein, “biological activity” encompasses one or more effector functions in common with those induced by action of NT-3, the native ligand of trkC, on a trkC-expressing cell. A “biological activity” of trkC can also encompass downstream signaling pathway(s) or effector functions that are different than those induced by action of NT-3. Without limitation, biological activities include any one or more of the following: ability to bind and activate trkC; ability to promote trkC receptor dimerization; the ability to promote the development, survival, function, maintenance and/or regeneration of damaged cells, in particular neurons in vitro or in vivo, including peripheral (sympathetic, sensory, and enteric) neurons, and central (brain and spinal cord) neurons, and non-neuronal cells, e.g. peripheral blood leukocytes. A particular preferred biological activity is the ability to treat (including prevention of) taxol-induced gut disorder, and/or repair and/or improve the function of a nerve cell damaged by taxol. Exemplary damaged neurons include any of sensory (including large-fiber sensory neurons), sympathetic, enteric, or central neurons e.g., dorsal root ganglia neurons, cranial ganglia neurons, and neurons from the spinal cord.

A “taxol-induced gut disorder” is a disorder of the gastrointestinal tract resulting from treatment with a chemotherapeutic agent taxol or other taxanes. As used herein, “taxol-induced gut disorder” refers to and includes any one or more symptoms associated with this disorder. In some embodiments, “taxol-induced gut disorder” is characterized by any of the following: abdominal distension, peritoneal ascites, intestinal hypertrophy (including hypertrophy of one or more of the stomach, large intestine, small intestine, caecum and/or colon), and decreased GI tract length. In some embodiments, “taxol-induced gut disorder” is characterized by intestinal obstruction(s) and/or stool softening or diarrhea. A “taxol-induced gut disorder” may include intestinal hypomotility or intestinal hypermotility and/or increased or decreased fluid absorption in the GI tract.

As used herein, “taxol” refers to paclitaxel (TAXOL®, Bristol-Myers Squibb Oncology, Princeton, N.J.), docetaxel (TAXOTERE®, Rhône-Poulenc Rorer, Antony, France), and other taxanes. Taxol (including other taxanes) may be administered either alone, or in combination with other drugs. Taxol is approved for and commonly used for treating various malignancies, including Kaposi's sarcoma and those of the breast, ovary, and lung. Taxol is also used to treat other malignancies of the prostate, head and neck, as well as various hematological malignancies. Taxol is also given during bone marrow transplants.

As used herein, “treatment” is an approach for obtaining beneficial or desired clinical results. For purposes of this invention, beneficial or desired clinical results include, but are not limited to, one or more of the following: alleviation of one or more symptoms associated with taxol-induced gut disorder; diminishment of extent of a taxol-induced gut disorder; stabilized (i.e., not worsening) state of a taxol-induced gut disorder; preventing occurrence or recurrence of a taxol-induced gut disorder; delaying the development of a taxol-induced gut disorder; delay or slowing of progression of a taxol-induced gut disorder; amelioration of a taxol-induced gut disorder; and remission (whether partial or total) of a taxol-induced gut disorder; increasing the rate of recovery from a taxol-induced gut disorder; reduction of incidence of a taxol-induced gut disorder and/or symptoms associated with a taxol-induced gut disorder.

“Palliating” a taxol-induced gut disorder or one or more symptoms of a taxol-induced gut disorder means lessening the extent and/or time course of undesirable clinical manifestations of a taxol-induced gut disorder in an individual or population of individuals treated with an agonist anti-trkC antibody in accordance with the invention.

“Reducing severity of a symptom” or “ameliorating a symptom” of a taxol-induced gut disorder means a lessening and/or improvement of one or more symptoms of a taxol-induced gut disorder as compared to not administering an agonist anti-trkC antibody. “Reducing severity” also includes shortening or reduction in duration of a symptom. Symptoms of a taxol-induced gut disorder are described supra.

As used herein, “delaying” development of a taxol-induced gut disorder means to defer, hinder, slow, retard, stabilize, and/or postpone development of the taxol-induced gut disorder. This delay can be of varying lengths of time, depending on the history of the taxol-induced gut disorder and/or individual being treated. As is evident to one skilled in the art, a sufficient or significant delay can, in effect, encompass prevention, in that the individual does not develop the taxol-induced gut disorder. A method that “delays” development of a taxol-induced gut disorder is a method that reduces probability of development of the gut disorder in a given time frame and/or reduces extent of the gut disorder in a given time frame, when compared to not using the method. Such comparisons are typically based on clinical studies, using a statistically significant number of subjects.

“Development” of a taxol-induced gut disorder means the onset and/or progression of a taxol-induced gut disorder within an individual. A taxol-induced gut disorder development can be detectable using standard clinical techniques as described herein. However, development also refers to disease progression that may be initially undetectable. For purposes of this invention, progression refers to the biological course of the disease state. “Development” includes occurrence, recurrence, and onset. As used herein “onset” or “occurrence” of a taxol-induced gut disorder includes initial onset and and/or recurrence.

An “effective amount” (in the taxol-induced gut disorder context) is an amount sufficient to effect beneficial or desired clinical results including, but not limited to, alleviating or reducing severity of one or more symptoms, or delaying the onset of the disease. An effective amount can be administered in one or more administrations. For purposes of this invention, an effective amount of an agonist anti-trkC antibody described herein is an amount sufficient to ameliorate, stabilize, reverse, slow and/or delay progression of or prevent taxol-induced gut disorder. An effective amount of an agonist anti-trkC antibody also encompasses an amount of an agonist anti-trkC antibody sufficient to enhance taxol treatment (therapeutic effect) of cancer (which can, in turn, mean that taxol dosage is increased and/or some other beneficial effect is observed such as reduction of side-effects of taxol treatment), as described herein. As is understood in the art, an effective amount of an agonist anti-trkC antibody may vary, depending on, inter alia, patient history as well as other factors such as the type (and/or dosage) of an agonist anti-trkC antibody used.

As used herein, administration “in conjunction” includes simultaneous administration and/or administration at different times. Administration in conjunction also encompasses administration as a co-formulation (e.g., an agonist anti-trkC antibody and taxol are present in the same composition) or administration as separate compositions. As used herein, administration in conjunction is meant to encompass any circumstance wherein an agonist anti-trkC antibody and taxol are administered to an individual, which can occur simultaneously and/or separately. As further discussed herein, it is understood that an agonist anti-trkC antibody and taxol can be administered at different dosing frequencies or intervals. For example, an agonist anti-trkC antibody can be administered weekly, while taxol can be administered less frequently. It is understood that the agonist anti-trkC antibody and taxol can be administered using the same route of administration or different routes of administration.

Taxol treatment is “enhanced” when an aspect of taxol treatment is improved (as compared to administering taxol without administering an agonist anti-trkC antibody). For example, the presence and/or intensity of undesired side-effects (such as gut disorder) may be reduced and/or eliminated in the presence of an agonist anti-trkC antibody relative to the presence and/or intensity of such side-effects in the absence of an agonist anti-trkC antibody.

An “individual” is a vertebrate, preferably a mammal, more preferably a human. Mammals include, but are not limited to, farm animals, sport animals, pets, primates, horses, cows, cats, dogs, and rodents (such as mice and rats).

As used herein, the singular form “a”, “an”, and “the” includes plural references unless indicated otherwise. For example, “an” antibody includes one or more antibodies and “a symptom” means one or more symptoms.

Methods of the Invention

With respect to all methods described herein, reference to agonist anti-trkC antibodies also includes compositions comprising one or more of these antibodies. These compositions may further comprise suitable excipients, such as pharmaceutically acceptable excipients including buffers, which are well known in the art.

Methods of Treating Taxol-Induced Gut Disorder Using Agonist Anti-TrkC Antibodies

The present invention encompasses methods of treating, preventing delaying the development of a symptom of and/or palliating taxol-induced gut disorder using agonist anti-trkC antibodies. The methods entail administering an effective amount of these antibodies to an individual in need thereof (various indications and aspects are described herein). An effective amount of the agonist anti-trkC antibody may be administered with or without other therapeutic agents. In some embodiments, the individual is human. However, the methods described are also applicable to the veterinary context (e.g., dogs, cats, cattle, horses).

Methods of assessing taxol-induced gut disorder are known in the art and described herein.

In one aspect, the invention provides methods for treating a taxol-induced gut disorder in an individual comprising administering an effective amount of agonist anti-trkC antibody.

As is evident, taxol can be administered before, during, or after treatment with agonist anti-trkC antibody, or can be delivered before initiating course of taxol therapy; during a course of taxol therapy, and/or after cessation of a course of taxol therapy. Administration can be before onset of gut disorder. Thus, in some embodiments, the individual is undergoing treatment with taxol. In other embodiments, the individual is undergoing treatment with taxol and cis-platinum. In still other embodiments, the individual has had prior taxol treatment.

Taxol is approved for and commonly used for treating various malignancies, including Kaposi's sarcoma and those of the breast, ovary, and lung. Taxol is also used to treat other malignancies, including those of the prostate, head and neck, and various hematological malignancies. Taxol is also given during bone marrow transplants. Accordingly, in one embodiment of the invention, the individual treated with agonist anti-trkC antibody has one or more of: breast cancer, lung cancer, ovarian cancer, Kaposi's sarcoma, prostate cancer, cancers of the head or neck, and hematological malignancies. In another embodiment, the individual treated with agonist anti-trkC antibody requires or has received a bone marrow transplant. In another embodiment, the individual has an indication (such as cancer) that is treatable with taxol.

Agonist Anti-TrkC Antibodies

Methods of the invention entail using anti-trkC antibodies that interact with trkC in a manner that activates trkC. Agonist anti-trkC antibodies are known in the art. See PCT WO 01/98361; Urfer et al. J. Biol. Chem. (1998) 273:5829-5840). Antibodies can encompass monoclonal antibodies, polyclonal antibodies, antibody fragments (e.g., Fab, Fab′, F(ab′)2, Fv, Fc, etc.), chimeric antibodies, single chain (ScFv), mutants thereof, fusion proteins comprising an antibody portion, and any other modified configuration of the immunoglobulin molecule that comprises an antigen recognition site of the required specificity. The antibodies may be murine, rat, human, or any other origin (including humanized antibodies). Thus, the agonist anti-trkC antibody may be a human antibody (such as antibody 1.6.4 (PCT WO 01/98361)) or may be a humanized antibody (including humanized monoclonal antibody 2256, see PCT WO 01/98361). In some embodiments, the agonist anti-trkC antibody binds human trkC. In some embodiments, the agonist anti-trkC antibody specifically binds human trkC. In some embodiments, the agonist anti-trkC antibody specifically binds a mammalian trkC (such as cat, dog, or horse). The agonist anti-trkC antibody may also bind human and rodent trkC. In one embodiment, the antibody is an antibody that recognizes one or more epitopes on human trkC extracellular domain. In another embodiment, the antibody is a mouse or rat antibody that recognizes one or more epitopes on human trkC extracellular domain. In some embodiments, the antibody binds human trkC and does not significantly bind trkC from another mammalian species (in some embodiments, vertebrate species). In some embodiments, the antibody binds human trkC as well as one or more trkC from another mammalian species (in some embodiments, vertebrate species). In another embodiment, the antibody recognizes one or more epitopes on a trkC selected from one or more of: primate, canine, feline, equine, and bovine. In some embodiments, the antibody binds trkC and does not significantly cross-react (bind) with other neurotrophin receptors (such as the related neurotrophin receptors, trkA and/or trkB). In some embodiments, the antibody binds trkC, and further binds trkA and/or trkB. In other embodiments, the antibody binds essentially the same trkC epitope as an antibody selected from any one or more of the following: 6.1.2, 6.4.1, 2345, 2349, 2.5.1, 2344, 2248, 2250, 2253, and 2256. See WO 01/98361. Examples of epitopes to which an antibody may be directed include but are not limited to domain V and/or domain IV of trkC. In another embodiment, the epitope includes one or more of the following residues: L284, E287, and N335 of human trkC. See Urfer et al. J. Biol. Chem. (1998) 273:5829-5840). In another embodiment, the epitope(s) can be continuous or discontinuous. In still other embodiment, the antibody does not trigger an unwanted or undesirable immune response, such as antibody-mediated lysis. See, e.g., PCT/GB99/01441; UK Patent Application No. 9809951.8. In some embodiments, the constant region comprises the human heavy chain IgG2a constant region containing the following mutations: A330P331 to S330S331 (amino acid numbering with reference to the wildtype IgG2a sequence; see Eur. J. Immunol. (1999) 29:2613-2624).

In some embodiments, the anti-trkC antibody is humanized (such as antibody A5 described herein). In some embodiments, the anti-trkC antibody is antibody A5 (as described herein). In other embodiments, the anti-trkC antibody comprises one or more CDR(s) of antibody A5 (such as one, two, three, four, five, or, in some embodiments, all six CDRs from A5). In some embodiments, the anti-trkC antibody comprises the heavy chain CDRs from antibody A5. In some embodiments, the anti-trkC antibody comprises the light chain CDRs from antibody A5. In some embodiments, the anti-trkC antibody comprises the heavy chain and the light chain CDRs from antibody A5. In some embodiments, the antibody is human. In still other embodiments, the anti-trkC antibody comprises the amino acid sequence of the heavy chain variable region shown in Table 3 (SEQ ID NO:1) and the amino acid sequence of the light chain variable region shown in Table 4 (SEQ ID NO:2). In still other embodiments, the anti-trkC antibody comprises the amino acid sequence of the heavy chain variable region shown in Table 3 (SEQ ID NO:1). In still other embodiments, the anti-trkC antibody comprises the amino acid sequence of the light chain variable region shown in Table 4 (SEQ ID NO:2). In still other embodiments, the antibody comprises a modified constant region, such as a constant region that is immunologically inert, e.g., does not trigger complement mediated lysis, or does not stimulate antibody-dependent cell mediated cytotoxicity (ADCC). In other embodiments, the constant region is modified as described in Eur. J. Immunol. (1999) 29:2613-2624; PCT Application No. PCT/GB99/01441; and/or UK Patent Application No. 9809951.8. In some embodiments, the constant region comprises the human heavy chain IgG2a constant region containing the following mutations: A330P331 to S330S331 (amino acid numbering with reference to the wildtype IgG2a sequence; see Eur. J. Immunol. (1999) 29:2613-2624). Anti-trkC antibodies are described in U.S. Ser. No. 60/532,592.

The anti-trkC antibody A5 is a humanized and affinity matured antibody which binds human and rodent trkC receptor. See, U.S. Ser. No. 60/532,592. The amino acid sequences of the heavy chain and light chain variable regions of A5 are shown in Table 3 (SEQ ID NO:1) and Table 4 (SEQ ID NO:2), respectively. The complementarity determining region (CDR) portions of antibody A5 (including Chothia and Kabat CDRs) are also diagrammatically depicted in Tables 3 and 4. The amino acid sequences of the individual extended CDRs of A5 are shown in Table 5.

TABLE 3

A5 heavy chain variable region amino acid
sequence. Kabat CDRs are shown as underlined
italics; Chothia CDRs are shown as bold.

| | |
|---|---|
| QVQLVQSGAEVKKPGASVKVSCKASGYTFTYRIHWV | (SEQ ID NO:1) |
| RQAPGQGLEWMGRVTMTRDT | |
| STSTVYMELSSLRSEDTAVYY | |
| WGQGTTVTVS | |

TABLE 4

A5 light chain variable region amino acid
sequence. Kabat CDRs are shown as underlined
italics; Chothia CDRs are shown as bold.

| | |
|---|---|
| DIQMTQSPSSLSASVGDRVTTTC | (SEQ ID NO:2) |
| WYQQKPGKAPKLLIYGVPSRFSGSGSGT | |
| DFTFTISSLQPEDIATYYCFGQGTKLEI | |
| KRT | |

TABLE 5
A5 extended CDRs

| | |
|---|---|
| A5 CDR H1 (extended CDR): | |
| KASGYTFTSYRIH | (SEQ ID NO:3) |
| A5 CDR H2 (extended CDR): | |
| EIYPSNARTNYNEKFKS | (SEQ ID NO:4) |
| A5 CDR H3 (extended CDR): | |
| CARKYYYGNTRRSWYFDV | (SEQ ID NO:5) |
| A5 CDR L1 (extended CDR): | |
| RASESIDNYGISFLA | (SEQ ID NO:6) |
| A5 CDR L2 (extended CDR): | |
| AASNRGS | (SEQ ID NO:7) |
| A5 CDR L3 (extended CDR): | |
| QQSKTVPRT | (SEQ ID NO:8) |

The following materials have been deposited with the American Type Culture Collection, 10801 University Boulevard, Manassas, Va., USA (ATCC):

| | | ATCC | |
|---|---|---|---|
| Material | | Accession No. | Date of Deposit |
| Eb.pur.2256.A5 | A5 light chain | PTA-5682 | Dec. 5, 2003 |
| Db.2256.A5 | A5 heavy chain | PTA-5683 | Dec. 5, 2003 |

Vector Eb.pur.2256.A5 is a polynucleotide encoding the A5 light chain variable region and the human light chain kappa constant region; and vector Db.2256.A5 is a polynucleotide encoding the A5 heavy chain variable region and the human heavy chain IgG2a constant region containing the following mutations: A330P331 to S330S331 (amino acid numbering with reference to the wildtype IgG2a sequence; see Eur. J. Immunol. (1999) 29:2613-2624). These polynucleotide sequences are shown in Tables 6 and 7 below and may be used to express A5 recombinantly.

TABLE 6
A5 Light chain full nucleotide sequence

(SEQ ID NO:9)

GATATCCAGATGACACAGTCCCCATCCTCCCTGTCTGCCTCTGTGGGTGA
CCGCGTCACCATCACCTGCCGCGCAAGTGAGAGCATCGACAACTATGGCA
TTTCCTTCCTGGCCTGGTATCAGCAGAAGCCGGGCAAAGCACCAAAACTC
CTGATCTATGCTGCATCCAATCGGGGTTCAGGTGTCCCATCACGCTTCAG
TGGCAGTGGCTCTGGTACAGATTTCACCTTCACCATTAGCAGCCTGCAAC
CAGAAGATATTGCCACTTATTACTGCCAACAGAGTAAGACTGTGCCACGC
ACTTTCGGTCAAGGCACCAAGCTGGAGATCAAACGCACTGTGGCTGCACC
ATCTGTCTTCATCTTCCCTCCATCTGATGAGCAGTTGAAATCCGGAACTG
CCTCTGTTGTGTGCCTGCTGAATAACTTCTATCCACGCGAGGCCAAAGTA
CAGTGGAAGGTGGATAACGCCCTCCAATCCGGTAACTCCCAGGAGAGTGT
CACAGAGCAGGACAGCAAGGACAGCACCTACAGCCTCAGCAGCACCCTGA
CCCTGAGCAAAGCAGACTACGAGAAACACAAAGTCTACGCCTGCGAAGCC
ACCCATCAGGGCCTGAGTTCTCCAGTCACAAAGAGCTTCAACCGCGGTGA
GTGC

TABLE 7

A5 heavy chain full nucleotide sequence (including modified
IgG2 as described herein)

(SEQ ID NO:10)

CAGGTGCAGCTGGTGCAGTCTGGTGCTGAGGTGAAGAAGCCTGGCGCTTC
CGTGAAGGTTTCCTGCAAAGCATCTGGTTACACCTTTACCAGCTATCGGA
TCCACTGGGTGCGCCAAGCCCCTGGTCAAGGCCTGGAGTGGATGGGCGAA
ATCTACCCAAGCAACGCGCGCACTAACTACAACGAGAAGTTCAAATCCCG
GGTGACCATGACTCGCGATACCTCCACCAGCACTGTCTACATGGAACTGA
GCTCTCTGCGCTCTGAGGACACTGCTGTGTATTACTGTGCCCGCAAGTAC
TATTACGGCAATACGCGTCGCTCCTGGTACTTCGATGTGTGGGGCCAGGG
TACCACTGTTACCGTGTCCTCTGCCTCCACCAAGGGCCCATCTGTCTTCC
CACTGGCCCCATGCTCCCGCAGCACCTCCGAGAGCACAGCCGCCCTGGGC
TGCCTGGTCAAGGACTACTTCCCAGAACCTGTGACCGTGTCCTGGAACTC
TGGCGCTCTGACCAGCGGCGTGCACACCTTCCCAGCTGTCCTGCAGTCCT
CAGGTCTCTACTCCCTCAGCAGCGTGGTGACCGTGCCATCCAGCAACTTC
GGCACCCAGACCTACACCTGCAACGTAGATCACAAGCCAAGCAACACCAA
GGTCGACAAGACCGTGGAGAGAAAGTGTTGTGTGGAGTGTCCACCTTGTC
CAGCCCCTCCAGTGGCCGGACCATCCGTGTTCCTGTTCCCTCCAAAGCCA
AAGGACACCCTGATGATCTCCAGAACCCCAGAGGTGACCTGTGTGGTGGT
GGACGTGTCCCACGAGGACCCAGAGGTGCAGTTCAACTGGTATGTGGACG
GAGTGGAGGTGCACAACGCCAAGACCAAGCCAAGAGAGGAGCAGTTCAAC
TCCACCTTCAGAGTGGTGAGCGTGCTGACCGTGGTGCACCAGGACTGGCT
GAACGGAAAGGAGTATAAGTGTAAGGTGTCCAACAAGGGACTGCCATCCA
GCATCGAGAAGACCATCTCCAAGACCAAGGGACAGCCAAGAGAGCCACAG
GTGTATACCCTGCCACCATCCAGAGAGGAGATGACCAAGAACCAGGTGTC
CCTGACCTGTCTGGTGAAGGGATTCTATCCATCCGACATCGCCGTGGAGT
GGGAGTCCAACGGACAGCCAGAGAACAACTATAAGACCACCCCTCCAATG
CTGGACTCCGACGGATCCTTCTTCCTGTATTCCAAGCTGACCGTGGACAA
GTCCAGATGGCAGCAGGGAAACGTGTTCTCTTGTTCCGTGATGCACGAGG
CCCTGCACAACCACTATACCCAGAAGAGCCTGTCCCTGTCTCCAGGAAAG

There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Chothia et al. (1989) Nature 342:877; Al-lazikani et al (1997) J. Molec. Biol. 273:927-948)). As used herein, a CDR may refer to CDRs defined by either approach or by a combination of both approaches. Identification of CDRs is well within the skill of the art.

In another embodiment, the anti-trkC agonist antibody comprises one or more CDR(s) of antibody A5 (such as one, two, three, four, five, or, in some embodiments, all six CDRs from A5). CDR(s) may be Kabat, Chothia, or a combination of Kabat and Chothia

The binding affinity of anti-trkC agonist antibody to trkC may be any of about 500 nM, 400 nM, 300 nM, 200 nM, 100 nM, about 50 nM, about 20 nM, about 10 nM, about 1 nM, about 500 pM, about 100 pM, or about 50 pM to any of about 2 pM, about 5 pM, about 10 pM, about 15 pM, about 20 pM, or about 40 pM. In some embodiments, the binding affinity is any of about 100 nM, about 50 nM, about 10 nM, about 1 nM, about 500 pM, about 100 pM, or about 50 pM, or less than about 50 pM. In some embodiments, the binding affinity is less than any of about 100 nM, about 50 nM, about 10 nM, about 1 nM, about 500 pM, about 100 pM, or about 50 pM. In still other embodiments, the binding affinity is about 2 pM, about 5 pM, about 10 pM, about 15 pM, about 20 pM, about 40 pM, or greater than about 40 pM. As is well known in the art, binding affinity can be expressed as K_{D}, or dissociation constant, and an increased binding affinity corresponds to a decreased K_{D}. The binding affinity of mouse anti-trkC agonist monoclonal antibody 2256 to human trkC is about 40 nM, as assessed using BIAcore analysis. The binding affinity of humanized anti-trkC agonist antibody A5 (described herein) to human trkC is about 0.28 nM, and to rat trkC is about 19 nM, as assessed using BIAcore analysis.

One way of determining binding affinity of antibodies to trkC is by measuring binding affinity of monofunctional Fab fragments of the antibody. To obtain monofunctional Fab fragments, an antibody (for example, IgG) can be cleaved with papain or expressed recombinantly. The affinity of an anti-trkC Fab fragment of an antibody can be determined by surface plasmon resonance (BIAcore3000™ surface plasmon resonance (SPR) system, BIAcore, INC, Piscaway N.J.). CM5 chips can be activated with N-ethyl-N′-(3-dimethylaminopropyl)-carbodiinide hydrochloride (EDC) and N-hydroxysuccinimide (NHS) according to the supplier's instructions. Human trkC-Fc fusion protein (“htrkC”) (or any other trkC, such as rat trkC) can be diluted into 10 mM sodium acetate pH 5.0 and injected over the activated chip at a concentration of 0.0005 mg/mL. Using variable flow time across the individual chip channels, two ranges of antigen density can be achieved: 200-400 response units (RU) for detailed kinetic studies and 500-1000 RU for screening assays. The chip can be blocked with ethanolamine. Regeneration studies have shown that a mixture of Pierce elution buffer (Product No. 21004, Pierce Biotechnology, Rockford, Ill.) and 4 M NaCl (2:1) effectively removes the bound Fab while keeping the activity of htrkC on the chip for over 200 injections. HBS-EP buffer (0.01M HEPES, pH 7.4, 0.15 NaCl, 3 mM EDTA, 0.005% Surfactant P20) is used as running buffer for the BIAcore assays. Serial dilutions (0.1-10× estimated K_{D}) of purified Fab samples are injected for 1 min at 100 μL/min and dissociation times of up to 2 h are allowed. The concentrations of the Fab proteins are determined by ELISA and/or SDS-PAGE electrophoresis using a Fab of known concentration (as determined by amino acid analysis) as a standard. Kinetic association rates (kₒₙ) and dissociation rates (k_{off}) are obtained simultaneously by fitting the data to a 1:1 Langmuir binding model (Karlsson, R. Roos, H. Fagerstam, L. Petersson, B. (1994). Methods Enzymology 6:99-110) using the BIAevaluation program. Equilibrium dissociation constant (K_{D}) values are calculated as k_{off}/kₒₙ.

In another aspect, antibodies (e.g., human, humanized, mouse, chimeric) that can activate human trkC receptor may be made by using immunogens which express one or more extracellular domains of trkC. One example of an immunogen is cells with high expression of trkC, which can be obtained as described herein. Another example of an immunogen that can be used is a soluble protein (such as a trkC immunoadhesin) which contains the extracellular domain or a portion of the extracellular domain of trkC receptor.

The route and schedule of immunization of the host animal are generally in keeping with established and conventional techniques for antibody stimulation and production, as further described herein. General techniques for production of human and mouse antibodies are known in the art and are described herein.

It is contemplated that any mammalian subject including humans or antibody producing cells therefrom can be manipulated to serve as the basis for production of mammalian, including human, hybridoma cell lines. Typically, the host animal is inoculated intraperitoneally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W. et al., (1982) In Vitro, 18:377-381. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the anti-trkC monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies specific for trkC, or a portion thereof.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a human or other species of trkC receptor, or a fragment of the human or other species of trkC receptor, or a human or other species of trkC receptor or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glytaradehyde, succinic anhydride, SOCl2, or R1N═C═NR, where R and R1 are different alkyl groups can yield a population of antibodies (e.g., monoclonal antibodies). Another example of an immunogen is cells with high expression of trkC, which can be obtained from recombinant means, or by isolating or enriching cells from a natural source that express a high level of trkC. These cells may be of human or other animal origin, and may be used as an immunogen as directly isolated, or may be processed in such that immunogenicity is increased, or trkC expression (of a fragment of trkC) is increased or enriched. Such processing includes, but is not limited to, treatment of the cells or fragments thereof with agents designed to increase their stability or immunogenicity, such as, e.g., formaldehyde, glutaraldehyde, ethanol, acetone, and/or various acids. Further, either before or after such treatment the cells may be processed in order to enrich for the desired immunogen, in this case trkC or fragment thereof. These processing steps can include membrane fractionation techniques, which are well known in the art.

If desired, the anti-trkC antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity, or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to trkC receptor and greater efficacy in activating trkC receptor. It will be apparent to one of skill in the art that one or more polynucleotide changes can be made to the anti-trkC antibody and still maintain its binding ability to trkC extracellular domain or epitopes of trkC.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; 6,180,370; 6,548,640; U.S. Ser. No. 10/745,775, and PCT/US03/41252. For example, the constant region may be engineered to more resemble human constant regions to avoid immune response if the antibody is used in clinical trials and treatments in humans. See, for example, U.S. Pat. Nos. 5,997,867 and 5,866,692. Humanization can also include affinity maturation. See, e.g., U.S. Ser. No. 10/745,775, and PCT/US03/41252.

In the recombinant humanized antibodies, the Fcγ portion can be modified to avoid interaction with Fcγ receptor and the complement immune system. PCT WO 99/58572.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. Nature 349:293-299 (1991), Lobuglio et al. Proc. Nat. Acad. Sci. USA 86:4220-4224 (1989), Shaw et al. J Immunol. 138:4534-4538 (1987), and Brown et al. Cancer Res. 47:3577-3583 (1987). Other references describe rodent CDRs grafted into a human supporting framework region (FR) prior to fusion with an appropriate human antibody constant domain. See, for example, Riechmann et al. Nature 332:323-327 (1988), Verhoeyen et al. Science 239:1534-1536 (1988), and Jones et al. Nature 321:522-525 (1986). Another reference describes rodent CDRs supported by recombinantly veneered rodent framework regions. See, for example, European Patent Publication No. 519,596. These “humanized” molecules are designed to minimize unwanted immunological response toward rodent anti-human antibody molecules which limits the duration and effectiveness of therapeutic applications of those moieties in human recipients. For example, the antibody constant region can be engineered such that it is immunologically inert, e.g., does not trigger complement mediated lysis or does not stimulate antibody-dependent cell mediated cytotoxicity (ADCC). In other embodiments, the constant region is modified as described in Eur. J. Immunol. (1999) 29:2613-2624. See, e.g. PCT/GB99/01441; UK patent application No. 9809951.8. Other methods of humanizing antibodies that may also be utilized are disclosed by Daugherty et al., Nucl. Acids Res. 19:2471-2476 (1991) and in U.S. Pat. Nos. 6,180,377; 6,054,297; 5,997,867; 5,866,692; 6,210671; 6,350,861; and PCT Publication No. WO 01/27160.

In yet another alternative, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Abgenix, Inc. (Fremont, Calif.) and HuMAb-Mouse®and TC Mouse™ from Medarex, Inc. (Princeton, N.J.).

In an alternative, antibodies may be made recombinantly and expressed using any method known in the art. In another alternative, antibodies may be made recombinantly by phage display technology. See, for example, U.S. Pat. Nos. 5,565,332; 5,580,717; 5,733,743 and 6,265,150; and Winter et al., Annu. Rev. Immunol. 12:433-455 (1994). Alternatively, the phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S. and Chiswell, David J., Current Opinion in Structural Biology 3, 564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al., J. Mol. Biol. 222:581-597 (1991), or Griffith et al., EMBO J. 12:725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” Marks, et al., Bio/Technol. 10:779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT patent application PCT WO 9306213, published Apr. 1, 1993). Unlike traditional humanization of rodent antibodies by CDR grafting, this technique provides completely human antibodies, which have no framework or CDR residues of rodent origin. It is apparent that although the above discussion pertains to humanized antibodies, the general principles discussed are applicable to customizing antibodies for use, for example, in dogs, cats, primates, equines and bovines.

Antibodies may be made recombinantly by first isolating the antibodies made from host animals, obtaining the gene sequence, and using the gene sequence to express the antibody recombinantly in host cells (e.g., CHO cells). Another method that may be employed is to express the antibody sequence in plants (e.g., tobacco), transgenic milk, or in other organisms. Methods for expressing antibodies recombinantly in plants or milk have been disclosed. See, for example, Peeters et al. (2001) Vaccine 19:2756; Lonberg, N. and D. Huszar (1995) Int. Rev. Immunol 13:65; and Pollock et al. (1999) J Immunol Methods 231:147. Methods for making derivatives of antibodies, e.g., humanized, single chain, etc. are known in the art.

The antibodies made either by immunization of a host animal or recombinantly should exhibit any one or more of the following characteristics: (a) binds to trkC receptor; (b) binds to one or more epitopes of trkC receptor; (c) binds to trkC receptor to activate trkC receptor and/or one or more downstream pathways mediated by the trkC signaling function; (d) binds to trkC receptor to activate trkC receptor and treat, prevent, reverse, or ameliorate one or more symptoms of taxol-induced gut disorder; (e) does not bind to and/or activate trkB or trkA; (f) displays favorable pharmacokinetic and bioavailability properties; (g) binds to trkC receptor to activate trkC receptor and

Immunoassays and flow cytometry sorting techniques such as fluorescence activated cell sorting (FACS) can also be employed to isolate antibodies that are specific for trkC.

The antibodies can be bound to many different carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

DNA encoding agonist anti-trkC antibodies may be isolated and sequenced, as is known in the art. See PCT WO 01/98361. Generally, the monoclonal antibody is readily isolated and sequenced using conventional procedures (e.g., by using oligonucleotide probes that are capable of binding specifically to genes encoding the heavy and light chains of the monoclonal antibodies). The hybridoma cells serve as a preferred source of such cDNA. Once isolated, the DNA may be placed into expression vectors (such as expression vectors disclosed in PCT WO 87/04462), which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT WO 87/04462. The DNA also may be modified, for example, by substituting the coding sequence for human heavy and light chain constant domains in place of the homologous murine sequences, Morrison et al., Proc. Nat. Acad. Sci. 81: 6851 (1984), or by covalently joining to the immunoglobulin coding sequence all or part of the coding sequence for a non-immunoglobulin polypeptide. In that manner, “chimeric” or “hybrid” antibodies are prepared that have the binding specificity of an anti-trkC monoclonal antibody herein. The DNA encoding the agonist anti-trkC antibody (such as an antigen binding fragment thereof) may also be used for delivery and expression of agonist anti-trkC antibody in a desired cell, as described here. DNA delivery techniques are further described herein.

Anti-trkC antibodies may be characterized using methods well-known in the art. For example, one method is to identify the epitope to which it binds, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an anti-trkC antibody binds. Epitope mapping is commercially available from various sources, for example, Pepscan Systems (Edelhertweg 15, 8219 PH Lelystad, The Netherlands). The epitope can be a linear epitope, i.e., contained in a single stretch of amino acids, or a conformational epitope formed by a three-dimensional interaction of amino acids that may not necessarily be contained in a single stretch. Peptides of varying lengths (e.g., at least 4-6 amino acids long) can be isolated or synthesized (e.g., recombinantly) and used for binding assays with an anti-trkC antibody. In another example, the epitope to which the anti-trkC antibody binds can be determined in a systematic screening by using overlapping peptides derived from the trkC extracellular sequence and determining binding by the anti-trkC antibody. According to the gene fragment expression assays, the open reading frame encoding trkC is fragmented either randomly or by specific genetic constructions and the reactivity of the expressed fragments of trkC with the antibody to be tested is determined. The gene fragments may, for example, be produced by PCR and then transcribed and translated into protein in vitro, in the presence of radioactive amino acids. The binding of the antibody to the radioactively labeled trkC fragments is then determined by immunoprecipitation and gel electrophoresis. Certain epitopes can also be identified by using large libraries of random peptide sequences displayed on the surface of phage particles (phage libraries). Other compositions suitable for the methods described herein are described in a later section.

Yet another method which can be used to characterize an anti-trkC antibody is to use competition assays with other antibodies known to bind to the same antigen, i.e., trkC extracellular domain to determine if the anti-trkC antibody binds to the same epitope as other antibodies. Competition assays are well known to those of skill in the art. Examples of antibodies useful in competition assays include the following: antibodies 6.1.2, 6.4.1, 2345, 2349, 2.5.1, 2344, 2248, 2250, 2253, and 2256. See PCT WO 01/98361.

Epitope mapping can also be performed using domain swap mutants as described in PCT WO 0198361. Generally, this approach is useful for anti-trkC antibodies that do not significantly cross-react with trkA or trkB. Domain-swap mutants of trkC can be made by replacing extracellular domains of trkC with the corresponding domains from trkB or trkA. The binding of each agonist anti-trkC antibody to various domain-swap mutants can be evaluated and compared to its binding to wild type (native) trkC using ELISA or other method known in the art. In another approach, alanine scanning can be performed. Individual residues of the antigen the trkC receptor, are systematically mutated to another amino acid (usually alanine) and the effect of the changes is assessed by testing the ability of the modified trkC to bind to antibody using ELISA or other methods known in the art.

Identification of Agonist Anti-TrkC Antibodies

Agonist antibodies may be identified using art-recognized methods, including one or more of the following methods. For example, the kinase receptor activation (KIRA) assay described in U.S. Pat. Nos. 5,766,863 and 5,891,650 may be used. This ELISA-type assay is suitable for qualitative or quantitative measurement of kinase activation by measuring the autophosphorylation of the kinase domain of a receptor protein tyrosine kinase (rPTK, e.g. trk receptor), as well as for identification and characterization of potential agonist or antagonists of a selected rPTK. The first stage of the assay involves phosphorylation of the kinase domain of a kinase receptor, in the present case a trkC receptor, wherein the receptor is present in the cell membrane of a eukaryotic cell. The receptor may be an endogenous receptor or nucleic acid encoding the receptor, or a receptor construct, may be transformed into the cell. Typically, a first solid phase (e.g., a well of a first assay plate) is coated with a substantially homogeneous population of such cells (usually a mammalian cell line) so that the cells adhere to the solid phase. Often, the cells are adherent and thereby adhere naturally to the first solid phase. If a “receptor construct” is used, it usually comprises a fusion of a kinase receptor and a flag polypeptide. The flag polypeptide is recognized by the capture agent, often a capture antibody, in the ELISA part of the assay. An analyte, such as a candidate agonist, is then added to the wells having the adherent cells, such that the tyrosine kinase receptor (e.g. trkC receptor) is exposed to (or contacted with) the analyte. This assay enables identification of agonist ligands for the tyrosine kinase receptor of interest (e.g. trkC). Following exposure to the analyte, the adhering calls are solubilized using a lysis buffer (which has a solubilizing detergent therein) and gentle agitation, thereby releasing cell lysate which can be subjected to the ELISA part of the assay directly, without the need for concentration or clarification of the cell lysate.

The cell lysate thus prepared is then ready to be subjected to the ELISA stage of the assay. As a first step in the ELISA stage, a second solid phase (usually a well of an ELISA microtiter plate) is coated with a capture agent (often a capture antibody) that binds specifically to the tyrosine kinase receptor, or, in the case of a receptor construct, to the flag polypeptide. Coating of the second solid phase is carried out so that the capture agent adheres to the second solid phase. The capture agent is generally a monoclonal antibody, but, as is described in the examples herein, polyclonal antibodies or other agents may also be used. The cell lysate obtained is then exposed to, or contacted with, the adhering capture agent so that the receptor or receptor construct adheres to (or is captured in) the second solid phase. A washing step is then carried out, so as to remove unbound cell lysate, leaving the captured receptor or receptor construct. The adhering or captured receptor or receptor construct is then exposed to, or contacted with, an anti-phosphotyrosine antibody which identifies phosphorylated tyrosine residues in the tyrosine kinase receptor. In the preferred embodiment, the anti-phosphotyrosine antibody is conjugated (directly or indirectly) to an enzyme which catalyses a color change of a non-radioactive color reagent. Accordingly, phosphorylation of the receptor can be measured by a subsequent color change of the reagent. The enzyme can be bound to the anti-phosphotyrosine antibody directly, or a conjugating molecule (e.g., biotin) can be conjugated to the anti-phosphotyrosine antibody and the enzyme can be subsequently bound to the anti-phosphotyrosine antibody via the conjugating molecule. Finally, binding of the anti-phosphotyrosine antibody to the captured receptor or receptor construct is measured, e.g., by a color change in the color reagent.

Following initial identification, the agonist activity of a candidate antibody can be further confirmed and refined by bioassays, known to test the targeted biological activities. For example, the ability of anti-trkC monoclonal antibodies to mimic the activity of NT-3 or activate trkC can be tested in the PC12 neurite outgrowth assay using PC12 cells transfected with full-length human trkC (Urfer et al., Biochem. 36: 4775-4781 (1997); Tsoulfas et al., Neuron 10: 975-990 (1993)) and can be confirmed in known animal models of neurodegerative disease. This assay measures the outgrowth of neurite processes by rat pheocytochroma cells (PC 12) in response to stimulation by appropriate ligands. These cells express endogenous trkA and are therefore responsive to NGF. However, they do not express endogenous trkC and are therefore transfected with trkC expression construct in order to elicit response to NT-3 and trkC agonists. After incubating the transfected cells with anti-trkC antibodies, cells with neurites exceeding 2 times the diameter of the cell are counted. Anti-trkC antibodies that stimulate neurite outgrowth in transfected PC12 cells demonstrate trkC agonist activity.

The activation of trkC may also be determined by using various specific neurons at specific stages of embryonic development. Appropriately selected neurons can be dependent on trkC activation for survival, and so it is possible to determine the activation of trkC by following the survival of these neurons in vitro. Addition of candidate antibodies to primary cultures of appropriate neurons will lead to survival of these neurons for a period of at least several days if the candidate antibodies activate trkC. This allows the determination of the ability of the candidate antibody to activate trkC. In one example of this type of assay, the trigeminal ganglion from an E11 mouse embryo is dissected, dissociated and the resultant neurons are plated in a tissue culture dish at low density. The candidate antibodies are then added to the media and the plates incubated for 24-48 hours. After this time, survival of the neurons is assessed by any of a variety of methods. Samples which received an agonist anti-trkC antibody will typically display an increased survival rate over samples which receive a control antibody, and this allows the determination of the presence of an agonist anti-trkC antibody. See, e.g., Buchman et al. (1993) Development 118(3):989-1001.

Agonist antibodies may be identified by their ability to activate downstream signaling in a variety of cell types that express trkC, either naturally or after transfection of DNA encoding trkC. This trkC may be human or other mammalian (such a rodent or primate) trkC. The downstream signaling cascade may be detected by changes to a variety of biochemical or physiological parameters of the trkC expressing cell, such as the level of protein expression or of protein phosphorylation of proteins or changes to the metabolic or growth state of the cell (including neuronal survival and/or neurite outgrowth, as described herein). Methods of detecting relevant biochemical or physiological parameters are known in the art.

Administration of Agonist Anti-TrkC Antibodies

Various formulations of agonist anti-trkC antibodies may be used for administration. In some embodiments, an agonist anti-trkC antibody may be administered neat. In some embodiments, an agonist anti-trkC antibody is administered in a composition comprising a pharmaceutically acceptable excipient. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

Agonist anti-trkC antibodies can be formulated for administration by injection (e.g., intraperitoneally, intravenously, subcutaneously, intramuscularly, etc.). Accordingly, the antibodies may be combined with pharmaceutically acceptable vehicles such as saline, Ringer's solution, dextrose solution, and the like. The particular dosage regimen, i.e., dose, timing and repetition, will depend on the particular individual and that individual's medical history. Generally, a dose of less than about 1 ug/kg body weigh, at least about 1 ug/kg body weight; at least about 2 ug/kg body weight, at least about 5 ug/kg body weight, at least about 10 ug/kg body weight, at least about 20 ug/kg body weight, at least about 50 ug/kg body weight, at least about 100 ug/kg body weight, at least about 200 ug/kg body weight, at least about 500 ug/kg body weight, at least about 1 mg/kg, body weight, at least about 2 mg/kg body weight, at least about 5 mg/kg body weight, at least about 10 mg/kg body weight, at least about 30 mg/kg body weight, or more (such as about 50 mg/kg, about 100 mg/kg, about 200 mg/kg or about 500 mg/kg) is administered.

Empirical considerations, such as the half-life, generally will contribute to the determination of the dosage. Antibodies which are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on maintaining an effective concentration of agonist anti-trkC antibody in the patient and suppression and/or amelioration and/or delay of one or more symptoms of taxol gut disorder. Alternatively, sustained continuous release formulations of agonist anti-trkC antibodies may be appropriate. Various formulations and devices for achieving sustained release are known in the art. Administration of an agonist anti-trkC antibody in accordance with the method in the present invention can be continuous or intermittent, depending, for example, upon the recipient's physiological condition, whether the purpose of the administration is therapeutic or prophylactic, and other factors known to skilled practitioners. The administration of an agonist anti-trkC antibody may be essentially continuous over a preselected period of time or may be in a series of spaced dose, e.g., either before, during, or after developing symptoms of taxol-induced gut disorder, before, during, before and after, during and after, or before, during, and after developing symptoms of taxol-induced gut disorder.

Generally, for administration of agonist anti-trkC antibodies, an initial candidate dosage can be about 2 mg/kg. For the purpose of the present invention, a typical daily dosage might range from about 30 μg/kg to 100 mg/kg or more, depending on the factors mentioned above. For repeated administrations over several days or longer, depending on the condition, the treatment is sustained until a desired suppression of disease symptoms occurs or until sufficient therapeutic levels are achieved to treat or prevent taxol-induced gut disorder. An exemplary dosing regimen comprises administering an initial dose of about 2 mg/kg, followed by a weekly maintenance dose of about 1 mg/kg of the trkC agonist antibody, or followed by a maintenance dose of about 1 mg/kg every other week.

In one embodiment, dosages for an antibody may be determined empirically in individuals who have been given one or more administration(s) of an agonist anti-trkC antibody that activates trkC receptor to treat a taxol-induced gut disorder. Individuals are given incremental dosages of an agonist anti-trkC antibody. To assess efficacy of agonist anti-trkC antibodies or fragments thereof, an indicator of taxol-induced gut disorder disease state can be followed as described herein.

Other formulations include suitable delivery forms known in the art including, but not limited to, carriers such as liposome. See, for example, Mahato et al. (1997) Pharm. Res. 14:853-859. Liposomal preparations include, but are not limited to, cytofectins, multilamellar vesicles and unilamellar vesicles.

The formulations to be used for in vivo administration must be sterile. This is readily accomplished by, for example, filtration through sterile filtration membranes. Therapeutic agonist anti-trkC antibody compositions are generally placed into a container having a sterile access port, for example, an intravenous solution bag or vial having a stopper pierceable by a hypodermic injection needle.

The agonist anti-trkC antibody is administered to a individual in accord with known methods, such as intravenous administration, e.g., as a bolus or by continuous infusion over a period of time, by intramuscular, intraperitoneal, subcutaneous, oral, or topical routes. Agonist anti-trkC antibody can also be administered by inhalation. Commercially available nebulizers for liquid formulations, including jet nebulizers and ultrasonic nebulizers are useful for administration. Liquid formulations can be directly nebulized and lyophilized powder can be nebulized after reconstitution. Alternatively, agonist anti-trkC antibody can be aerosolized using a fluorocarbon formulation and a metered dose inhaler, or inhaled as a lyophilized and milled powder.

In some embodiments, more than one antibody may be present. The antibodies can be the same or different from each other. Such antibodies may contain at least one, at least two, at least three, at least four, at least five different antibodies. Preferably those antibodies have complementary activities that do not adversely affect each other.

A polynucleotide encoding an agonist anti-trkC antibody (such as an antigen binding fragment thereof) may also be used for delivery and expression of agonist anti-trkC antibody in a desired cell. It is apparent that an expression vector can be used to direct expression of an agonist anti-trkC antibody. The expression vector can be administered intraperitoneally, intravenously, intramuscularly, subcutaneously, intrathecally, intraventricularly, orally, enterally, parenterally, intranasally, dermally, or by inhalation. For example, administration of expression vectors includes local or systemic administration, including injection, oral administration, particle gun or catheterized administration, and topical administration. One skilled in the art is familiar with administration of expression vectors to obtain expression of an exogenous protein in vivo. See, e.g., U.S. Pat. Nos. 6,436,908; 6,413,942; and 6,376,471.

Targeted delivery of therapeutic compositions comprising a polynucleotide encoding an agonist anti-trkC antibody can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740; 4,777,127; GB Patent No. 2,200,651; and EP 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859; Liposomes that can act as gene delivery vehicles are described in U.S. Pat. No. 5,422,120; PCT Publication Nos. WO 95/13796; WO 94/23697; WO 91/14445; and EP 0 524 968. Additional approaches are described in Philip, Mol. Cell Biol. (1994) 14:2411, and in Woffendin, Proc. Natl. Acad. Sci. (1994) 91:1581.

Taxol (including other taxanes) may be administered either alone, or in conjunction with other drugs. Most commonly, taxol is delivered in a formulation comprising ethanol and cremophor EL™, which is diluted into an aqueous salt solution for treatment. Taxol may also be formulated in various other configurations, such as in emulsions. Taxol is commonly administered in conjunction with radiotherapy and/or other chemotherapeutic drugs, including, but not limited to, various platinum containing compounds (cis-platinum, carboplatinum, oxaliplatin), ifosfamide, 5-fluorouracil, doxorubicin, epirubicin, cyclophosphamide, gemcitabine, capecitabine, exisulind, topotecan, etoposide, vinca alkaloids (vincristine, vinblatin, and vinorelbine), and others. Taxol is also administered in conjunction with drugs designed to counteract or treat the adverse side effects of taxol and/or other chemotherapeutic agents that are administered in combination with taxol. For example, drugs such as erythropoietin (Epoiten, Darbopoiten), G-CSF, and GM-CSF) can be administered in conjunction with taxol to treat the hematological effects of chemotherapeutic agents. As another example, drugs such as phenothiazines (Compazine), Zofran, and Anzemet can be administered in conjunction with taxol to treat the nausea which often accompanies the use of chemotherapeutic agents. Patients can be pre-medicated before taxol treatment.

Taxol is approved for and commonly used for treating various malignancies, including Kaposi's sarcoma and those of the breast, ovary, and lung. Taxol is also used to treat other malignancies, including those of the prostate, head and neck, and various hematological malignancies. Taxol is also given during bone marrow transplants, e.g., to mobilize stem cells prior to bone marrow treatment. Representative dose regimens of taxol include: 135 mg/m² or 175 mg/m² administered intravenously over 3 hours every three weeks (for ovarian carcinoma); 175 mg/m² administered intravenously over 3 hours every three weeks (for breast carcinoma); 135 mg/m² administered intravenously over 24 hours (for non-small cell lung carcinoma); 135 mg/m² administered intravenously over 3 hours every three weeks or 100 mg/m² administered intravenously over 3 hours every two weeks (Kaposi's sarcoma). See also Taxol Prescribing Information (product insert), Bristol Meyers Squibb (1998) (available at http://www.taxol.com/txpi.html). In other embodiments, taxol is administered at 775 mg/m², 475 mg/m², 200 mg/m², and/or 350 mg/m². Taxol is also used at high doses during bone marrow transplant, for example, at doses as high as 825 mg/m². In some embodiments, taxol treatment during bone marrow transplantation is in conjunction with one or more of the following agents: melphalan, cyclophosphamide, thiotepa and carboplatin. See, e.g., Vahdat et al (2002) Bone Marrow Transplant 30(3):149-153.

The agonist anti-trkC antibody may be administered in conjunction with the taxol, i.e., administered in combination with, in concert with, or sequentially with taxol. Such administration includes administering the antibody to the patient prior to administration of the gut-disorder-inducing drug or administration of the antibody to the patient after the administration of the gut-disorder-inducing drug. Administration in conjunction, as used herein, comprises simultaneous administration and/or administration at different times. Administration in conjunction also encompasses administration as a co-formulation (i.e., the agonist anti-trkC antibody and taxol are present (combined) in the same composition) and/or administration as separate compositions. As used herein, “administration in conjunction” is meant to encompass any circumstance wherein agonist anti-trkC antibody and taxol are administered in an effective amount to an individual. As further discussed herein, it is understood that the agonist anti-trkC antibody and taxol can be administered at different dosing frequencies and/or intervals. For example, an agonist anti-trkC antibody may be administered weekly, while taxol may be administered more frequently. It is understood that an agonist anti-trkC antibody and taxol can be administered using the same route of administration or different routes of administration, and that different dosing regimens may change over the course of administration(s). Administration may be before the onset of gut disorder.

The treatment of cancer with taxol can also be enhanced as described herein, by administration of the taxol in conjunction with an agonist anti-trkC antibody. The relative amounts and ratios of agonist anti-trkC antibody and taxol may vary. In some embodiments, enough agonist anti-trkC antibody will be administered so as to allow an reduction of undesired side-effects (such as gut disorder) induced by or associated with taxol treatment.

Methods of Assessing Efficacy of Treatment with Agonist Anti-TrkC Antibodies

Assessment and diagnosis of taxol-induced gut disorder is well known in the art. Assessment of treatment efficacy can be performed on several different levels. Assessment may be made by monitoring clinical signs, as is known in the art. In some embodiments, taxol-induced gut disorder is characterized by any of the following symptoms: abdominal distension, peritoneal ascites, intestinal hypertrophy (including hypertrophy of one or more of the stomach, large intestine, small intestine, caecum and/or colon), decreased GI tract motility and decreased GI tract length. In some embodiments, “taxol-induced gut disorder” is characterized by intestinal obstruction(s) and/or stool softening or diarrhea

Compositions for Use in Treatment of Taxol-Induced Gut Disorder

The invention also provides compositions for use in any of the methods described herein. The compositions used in the methods of the invention comprise an effective amount of an agonist anti-trkC antibody. Examples of such compositions, as well as how to formulate, are also described in an earlier section and below. The composition used in the present invention can further comprise pharmaceutically acceptable carriers, excipients, or stabilizers (Remington: The Science and practice of Pharmacy 20th Ed. (2000) Lippincott Williams and Wilkins, Ed. K. E. Hoover.), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

In one aspect, the invention provides compositions comprising an agonist anti-trkC antibody. In other embodiments, the agonist anti-trkC antibody recognizes human trkC. In still other embodiments, the agonist anti-trkC antibody is humanized (such as antibody A5 described herein). In other embodiments, the anti-trkC agonist antibody comprises one or more CDR(s) of antibody A5 (such as one, two three, four, five or, in some embodiments, all six CDRs from A5). In still other embodiments, the anti-trkC agonist antibody comprises the amino acid sequence of the heavy chain variable region shown in Table 3 (SEQ ID NO:1) and the amino acid sequence of the light chain variable region shown in Table 4 (SEQ ID NO:2). In still other embodiments, the agonist anti-trkC antibody is a human antibody.

It is understood that the compositions can comprise more than one agonist anti-trkC antibody (e.g., a mixture of agonist anti-trkC antibodies that recognize different epitopes of trkC). Other exemplary compositions comprise more than one agonist anti-trkC antibody that recognize the same epitope(s), or different species of agonist anti-trkC antibodies that bind to different epitopes of trkC.

The agonist anti-trkC antibody and compositions thereof can also be used in conjunction with other agents that serve to enhance and/or complement the effectiveness of the agonist anti-trkC antibody. For example, such additional compounds may include compounds known to be useful for the treatment of taxol-induced gut disorder or side effects of taxol treatment, e.g. anemia or nausea, including but not limited to: erythropoietin (Epoiten, Darbopoiten) G-CSF, and GM-CSF, phenothiazines (Compazine), Zofran, and Anzemet. Such molecules are suitably present in combination in amounts that are effective for the purpose intended. The trkC agonist antibody and compositions thereof can also be used in conjunction with other agents that serve to enhance and/or complement the effectiveness of the antibodies, including erythropoietin (Epoiten, Darbopoiten) G-CSF, and GM-CSF, phenothiazines (Compazine), Zofran, and Anzemet.

In other embodiments, the invention provides compositions (described herein) for use in any of the methods described herein, whether in the context of use as a medicament and/or use for manufacture of a medicament.

Kits

The invention also provides kits for use in the instant methods. Kits of the invention include one or more containers comprising an anti-trkC agonist antibody and instructions for use in accordance with any of the methods of the invention described herein. In some embodiments, these instructions comprise a description of selecting an individual suitable for treatment based on identifying whether that individual has a taxol-induced gut disorder and/or is at risk of developing taxol-induced gut disorder, and may further describe administration of the trkC agonist antibody for treatment and/or prevention of the gut disorder.

Thus, in one embodiment, the invention provides kits comprising an agonist anti-trkC antibody. The kit may further comprise instructions comprising a description of administering an agonist anti-trkC antibody to an individual at risk of developing taxol-induced gut disorder. In some embodiments, the invention provides kits for use with the methods described herein comprising an agonist anti-trkC antibody. In still other embodiments, the instructions comprise description of administering taxol in conjunction with an agonist anti-trkC antibody.

The kits of this invention are in suitable packaging. Suitable packaging includes, but is not limited to, vials, bottles, jars, flexible packaging (e.g., sealed Mylar or plastic bags), and the like. In some embodiments, the kit comprises a container and a label or package insert(s) on or associated with the container. The label or package insert indicates that the composition is useful for treating, preventing or ameliorating taxol-induced gut disorder. Instructions may be provided for practicing any of the methods described herein. The container holds a composition which is effective for treating taxol-induced gut disorder, and may have a sterile access port (for example the container may be an intravenous solution bag or a vial having a stopper pierceable by a hypodermic injection needle). At least one active agent in the composition is an trkC agonist antibody. The container may further comprise a second pharmaceutically active agent. Kits may optionally provide additional components such as buffers and interpretive information.

In some embodiments, the invention provides articles of manufacture comprising contents of the kits described above. In some embodiments, the kits comprise an agonist anti-trkC antibody with information indicating use to treat a taxol-induced gut disorder.

The following Examples are provided to illustrate but not limit the invention.

### EXAMPLE

### Example 1

### Development of a Taxol-Induced Gut Disorder in a Mouse Model, and Effect of Agonist Anti-trkC Monoclonal Antibodies on Taxol-Induced Gut Disorder

This example demonstrates the development of a taxol-induced gut disorder in a mouse model, and that treatment with anti-trkC agonist antibody ameliorates the development of the taxol-induced gut disorder.

Female Swiss-Webster mice, eight weeks old were treated with vehicle alone (control), taxol alone, or agonist anti-trkC mouse monoclonal antibody 2256 (PCT WO 01/98361; Urfer et al. J. Biol. Chem. (1998) 273:5829-5840)) and taxol. Taxol was obtained from Calbiochem and prepared as a 20 mg/kg solution in Cremophor/ethanol (50:50 w/w), which was diluted with saline (1 part Cremophor/ethanol to four parts saline vol:vol), filtered through a 0.1 micron filter and administered intraperitoneally (“IP”) within 30 minutes of preparation. All treatment animals received one round of three taxol or control (vehicle) treatments every other day for three doses, i.e., day 1, 3 and 5 of the protocol. The total taxol dose administered to each animal was split evenly into the three doses described above. Taxol dose was determined on a mg/m² basis, using the formula that body surface area (in square cm) equals 10.5 times the weight in grams to the two-thirds power. Specific taxol doses are described in the discussion of specific experiments, below.

Animals treated with mouse anti-trkC agonist antibody 2256 were dosed weekly, subcutaneously, starting on day 1 and lasting throughout the course of the protocol. Specific antibody doses are described in the discussion of specific experiments, below.

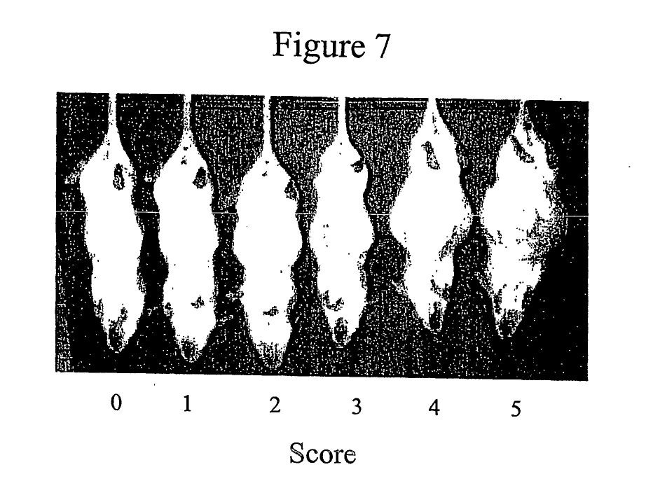

Taxol-induced gut disorder was assessed by measuring degree and quality of abdominal distension and intestinal hypertrophy after lifting the individual mouse by the tail. See FIG. 7. Degree and quality of taxol-induced abdominal distension was scored by a blinded observer using the following five point scale:
0-no noticeable abdominal bulge
1-slight bulge
2-distinct bulge (may be full stomach)
3-fat mouse
4-definite lumpy abdomen
5-swollen abdomen

FIG. 7 shows a photograph of exemplary animals that were rated 1-5, respectively.

Intestinal hypertrophy was assessed by measuring weight and length of the gastrointestinal (“GI”) tract as follows: animals were sacrificed, and the weight and length of the GI tract was assessed as follows: the entire GI tract was removed, emptied of any contents, and weighed. The GI tract was then separated into stomach, small intestine, and caecum plus large intestine and those parts were individually weighed.

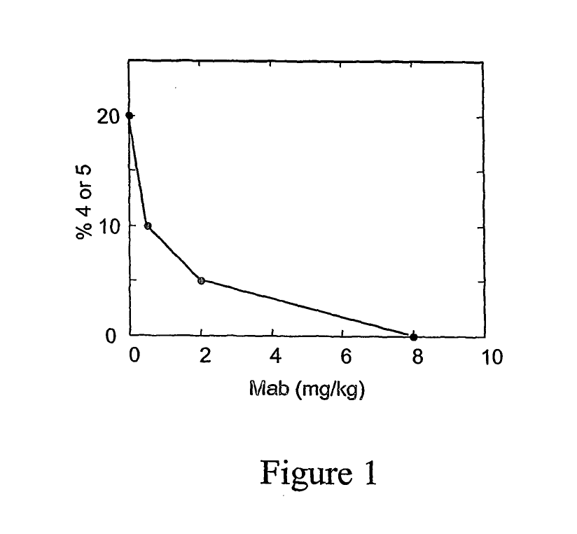

In a first experiment, animals were dosed with taxol at 300 mg/m² or vehicle (cremaphor:ethanol control), and taxol-treated and control animals were further dosed with zero, 0.5, 2, or 8 mg/kg of the mouse monoclonal trkC agonist Mab 2256, as shown in table 1. Degree and quality of abdominal distension was scored at three weeks. The results are shown in FIG. 1. Results are shown as the percentage of animals that scored either 4 or 5.

TABLE 1
Number of mice undergoing each treatment

| Ab dose | | Vehicle | 300 mg/m² |
|---|---|---|---|
| 0 | | 10 | 45 |
| 0.5 | mg/kg | 10 | 20 |
| 2 | mg/kg | 10 | 20 |
| 8 | mg/kg | 10 | 20 |

As shown in FIG. 1, treatment with taxol resulted in development of grade four and five abdominal distension in 20% of animals, and treatment with anti-trkC agonist antibody ameliorated the taxol-induced abdominal distension. By contrast, no control (vehicle-treated) animals developed grade four or five abdominal distension.

Selected animals were sacrificed and subjected to autopsy. Production of ascites in the peritoneum was noted in animals with abdominal distension (data not shown). Ascites production was not observed in control animals. Some taxol-treated animals exhibited intestinal obstruction(s) and/or stool softening or diarrhea.

A second experiment assessed both taxol-induced abdominal distension and taxol-induced intestinal hypertrophy. As shown in Table 2, animals were dosed with 225, 300, 375, or 450 mg/m² of taxol or vehicle, and 0 or 2 mg/kg of anti-trkC agonist mouse antibody Mab 2256. Animals were scored for gut distension weekly for six weeks, except that the animals treated with 450 mg/m² of taxol were sacrificed at four weeks and were thus scored for only 4 weeks. Results are shown as percentage of animals with grade four or five gut distension.

TABLE 2
Number of mice undergoing each treatment

| | | 225 | 300 | 375 | 450 |
|---|---|---|---|---|---|
| Ab conc. | Vehicle | mg/m² | mg/m² | mg/m² | mg/m² |
| No Ab | 10 | 10 | 15 | 15 | 20 |
| 2 mg/kg | 10 | 10 | 15 | 15 | 20 |

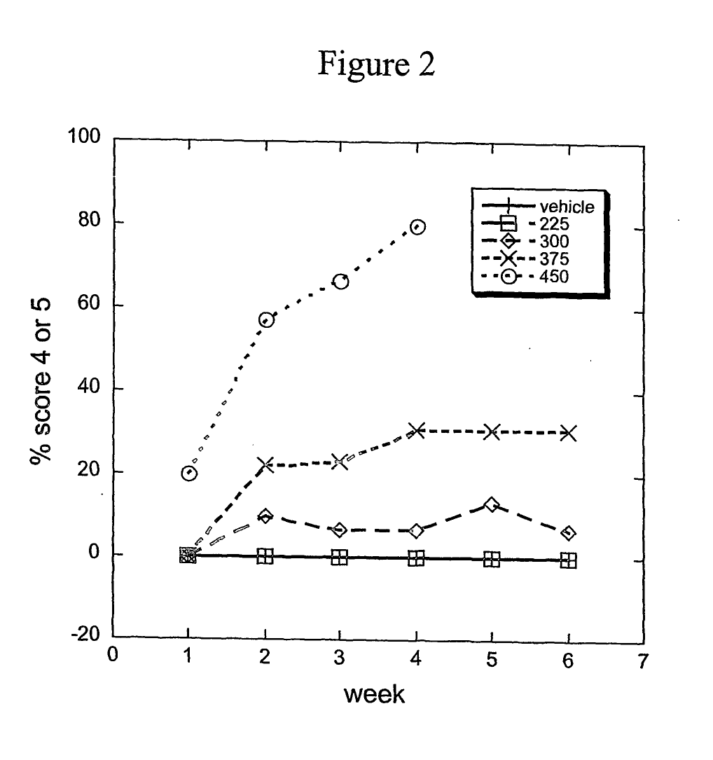

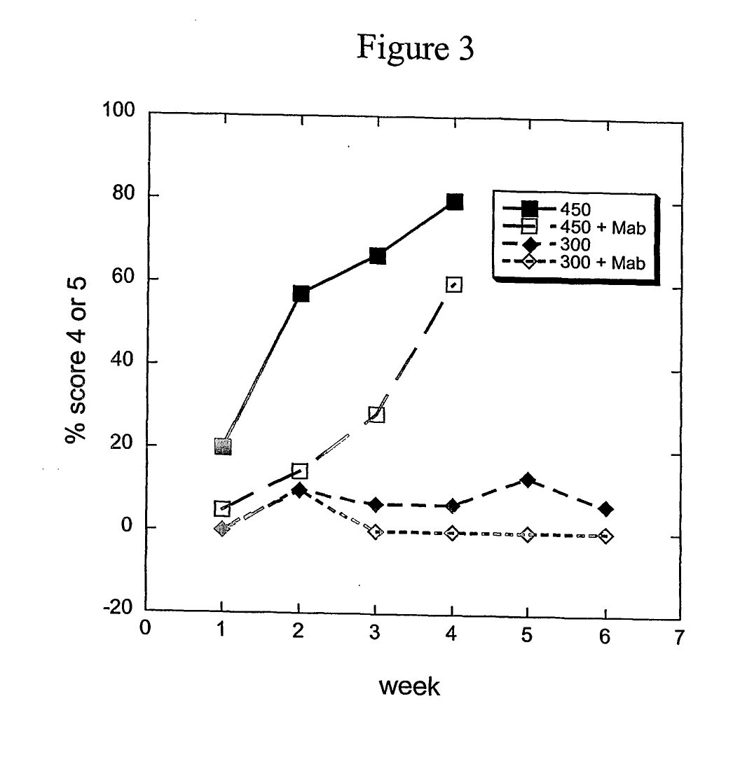

As shown in FIG. 2, treatment with 300, 375 or 450 mg/m² of taxol resulted in gut distension. As shown in FIG. 3, treatment with anti-trkC agonist antibody ameliorated gut distension in animals treated with 300 or 450 mg/m² of taxol.

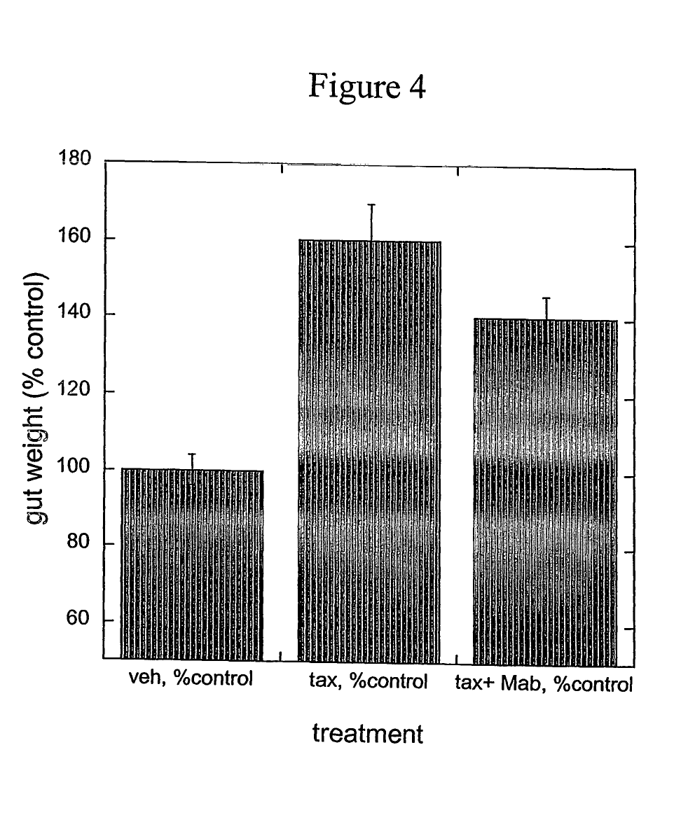

The animals treated with 450 mg/m² of taxol were sacrificed at four weeks and the animals treated with 375 mg/m² of taxol were sacrificed at six week. Intestinal hypertrophy was assessed as described above. Production of ascites in the peritoneum was noted in animals with abdominal distension. Ascites production was not observed in control animals. Some taxol-treated animals exhibited intestinal obstruction(s) and/or stool softening or diarrhea. The results of these analyses are shown in FIGS. 4, 5, 6, 8, and 9.

FIG. 4 shows that there was a significant increase in the weight of the GI tract from stomach through colon in animals treated with taxol at 450 mg/m², and this was partially prevented by treatment with Mab.

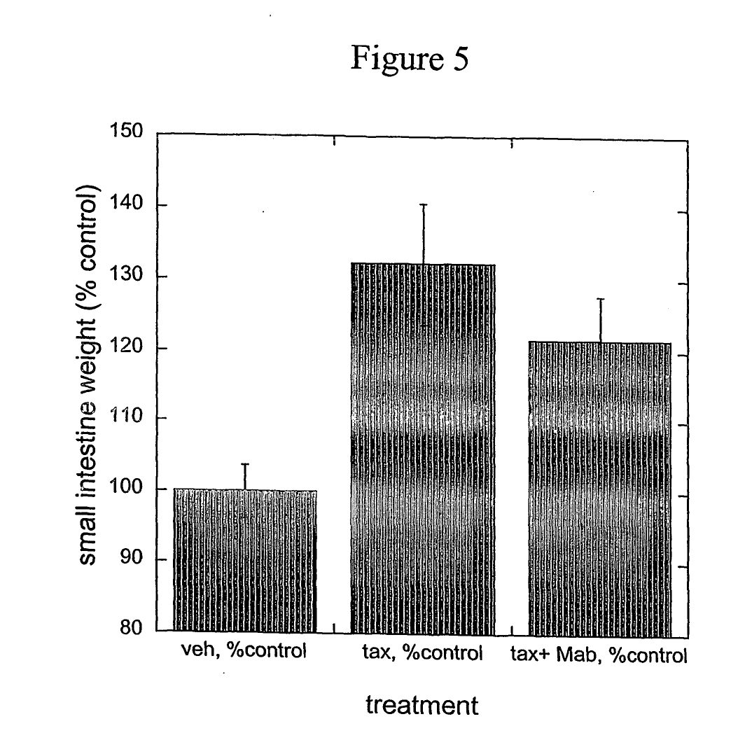

FIG. 5 shows that there was a significant increase in the weight of the small intestine in animals treated with taxol at 450 mg/m², and this was partially ameliorated by treatment with Mab.

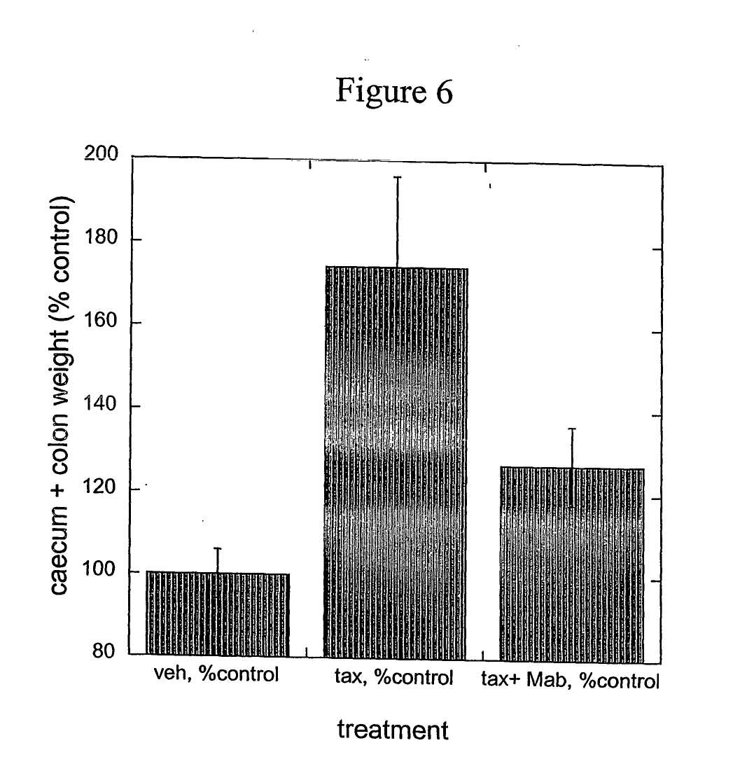

FIG. 6 shows that there was a significant increase in the weight of the caecum plus large intestine in animals treated with taxol at 450 mg/m², and this was ameliorated by treatment with Mab.

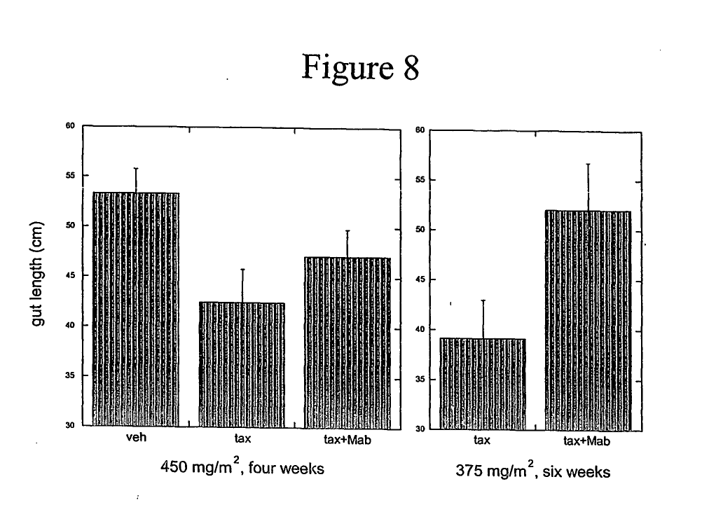

FIG. 8 shows that treatment with taxol at either 450 mg/m² or 375 mg/m² (four weeks or six weeks after treatment) caused a significant decrease in the length of the GI tract, and this was ameliorated by treatment with Mab 2256.

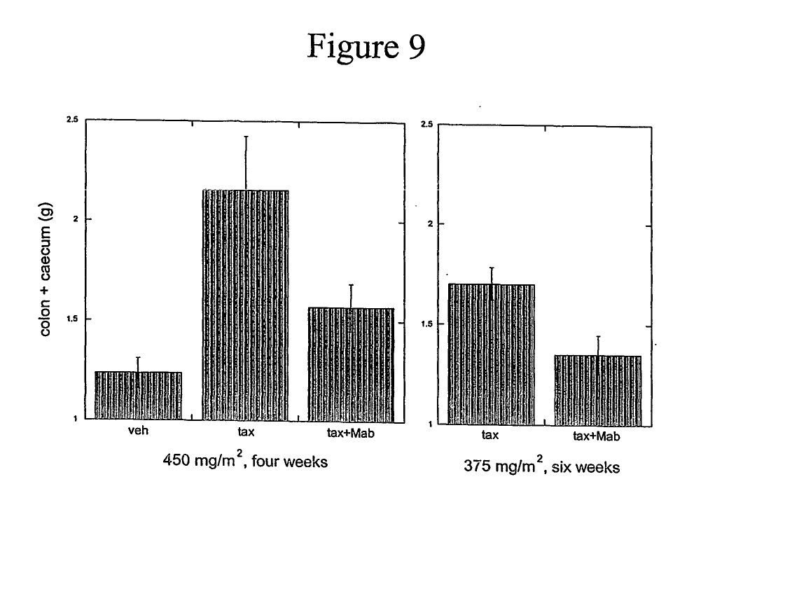

FIG. 9 shows that treatment with taxol at either 450 mg/m² or 375 mg/m² (four weeks or six weeks after treatment) caused a significant increase in the raw weight of the caecum plus large intestine, and this was ameliorated by treatment with Mab 2256.

Although the foregoing invention has been described in some detail by way of illustration and example for purposes of clarity of understanding, it will be apparent to those skilled in the art that certain changes and modifications may be practiced. Therefore, the descriptions and examples should not be construed as limiting the scope of the invention.

We claim:

1. A method for treating a taxol-induced gut disorder in a mammal comprising administering to the mammal an effective amount of an agonist anti-trkC antibody.

2. The method of claim 1, wherein the agonist anti-trkC antibody binds human trkC.

3. The method of claim 1, wherein the agonist anti-trkC antibody binds human trkC and rodent trkC.

4. The method of claim 1, wherein the agonist anti-trkC antibody binds an epitope in domain 5 of trkC.

5. The method of claim 4, wherein the trkC is human trkC.

6. The method of claim 1, wherein the agonist anti-trkC antibody is a human antibody.

7. The method of claim 1, wherein the agonist anti-trkC antibody is a humanized antibody.

8. The method of claim 1, wherein the agonist anti-trkC antibody is a monoclonal antibody.

9. The method of claim 1, wherein the agonist anti-trkC antibody comprises CDRs from heavy chain variable region shown in SEQ ID NO:1.

10. The method of claim 1, wherein the agonist anti-trkC antibody comprises CDRs from light chain variable region shown in SEQ ID NO:2.

11. The method of claim 1, wherein the agonist anti-trkC antibody comprises CDRs from heavy chain variable region shown in SEQ ID NO:1 and CDRs from light chain variable region shown in SEQ ID NO:2.

12. The method of claim 1, wherein the agonist anti-trkC antibody comprises the heavy chain variable region shown in SEQ ID NO:1 and the light chain variable region shown in SEQ ID NO:2.

13. A pharmaceutical composition for treating a taxol-induced gut disorder comprising an effective amount of an agonist anti-trkC antibody and a pharmaceutically acceptable carrier.

14. A kit for treating a taxol-induced gut disorder comprising an agonist anti-trkC antibody and an instruction for administering the agonist anti-trkC antibody to treat the taxol-induced gut disorder.


SEQUENCE LISTING (10 sequences)

SEQ ID NO: 1 - protein, 123 aa, Artificial Sequence
  Synthetic Construct
QVQLVQSGAEVKKPGASVKVSCKASGYTFTSYRIHWVRQAPGQGLEWMGEIYPSNARTNYNEKFKSRVTMTRDTSTSTVYMELSSLRSEDTAVYYCARKYYYGNTRRSWYFDVWGQGTTVTVS

SEQ ID NO: 2 - protein, 113 aa, Artificial Sequence
  Synthetic Construct
DIQMTQSPSSLSASVGDRVTITCRASESIDNYGISFLAWYQQKPGKAPKLLIYAASNRGSGVPSRFSGSGSGTDFTFTISSLQPEDIATYYCQQSKTVPRTFGQGTKLEIKRT

SEQ ID NO: 3 - protein, 13 aa, Artificial Sequence
  Synthetic Construct
KASGYTFTSYRIH

SEQ ID NO: 4 - protein, 17 aa, Artificial Sequence
  Synthetic Construct
EIYPSNARTNYNEKFKS

SEQ ID NO: 5 - protein, 18 aa, Artificial Sequence
  Synthetic Construct
CARKYYYGNTRRSWYFDV

SEQ ID NO: 6 - protein, 15 aa, Artificial Sequence
  Synthetic Construct
RASESIDNYGISFLA

SEQ ID NO: 7 - protein, 7 aa, Artificial Sequence
  Synthetic Construct
AASNRGS

SEQ ID NO: 8 - protein, 9 aa, Artificial Sequence
  Synthetic Construct
QQSKTVPRT

SEQ ID NO: 9 - DNA, 654 bp, Artificial Sequence
  Synthetic Construct
gatatccagatgacacagtccccatcctccctgtctgcctctgtgggtgaccgcgtcaccatcacctgccgcgcaagtgagagcatcgacaactatggcatttccttcctggcctggtatcagcagaagccgggcaaagcaccaaaactcctgatctatgctgcatccaatcggggttcaggtgtcccatcacgcttcagtggcagtggctctggtacagatttcaccttcaccattagcagcctgcaaccagaagatattgccacttattactgccaacagagtaagactgtgccacgcactttcggtcaaggcaccaagctggagatcaaacgcactgtggctgcaccatctgtcttcatcttccctccatctgatgagcagttgaaatccggaactgcctctgttgtgtgcctgctgaataacttctatccacgcgaggccaaagtacagtggaaggtggataacgccctccaatccggtaactcccaggagagtgtcacagagcaggacagcaaggacagcacctacagcctcagcagcaccctgaccctgagcaaagcagactacgagaaacacaaagtctacgcctgcgaagccacccatcagggcctgagttctccagtcacaaagagcttcaaccgcggtgagtgc

SEQ ID NO: 10 - DNA, 1350 bp, Artificial Sequence
  Synthetic Construct
caggtgcagctggtgcagtctggtgctgaggtgaagaagcctggcgcttccgtgaaggtttcctgcaaagcatctggttacacctttaccagctatcggatccactgggtgcgccaagcccctggtcaaggcctggagtggatgggcgaaatctacccaagcaacgcgcgcactaactacaacgagaagttcaaatcccgggtgaccatgactcgcgatacctccaccagcactgtctacatggaactgagctctctgcgctctgaggacactgctgtgtattactgtgcccgcaagtactattacggcaatacgcgtcgctcctggtacttcgatgtgtggggccagggtaccactgttaccgtgtcctctgcctccaccaagggcccatctgtcttcccactggccccatgctcccgcagcacctccgagagcacagccgccctgggctgcctggtcaaggactacttcccagaacctgtgaccgtgtcctggaactctggcgctctgaccagcggcgtgcacaccttcccagctgtcctgcagtcctcaggtctctactccctcagcagcgtggtgaccgtgccatccagcaacttcggcacccagacctacacctgcaacgtagatcacaagccaagcaacaccaaggtcgacaagaccgtggagagaaagtgttgtgtggagtgtccaccttgtccagcccctccagtggccggaccatccgtgttcctgttccctccaaagccaaaggacaccctgatgatctccagaaccccagaggtgacctgtgtggtggtggacgtgtcccacgaggacccagaggtgcagttcaactggtatgtggacggagtggaggtgcacaacgccaagaccaagccaagagaggagcagttcaactccaccttcagagtggtgagcgtgctgaccgtggtgcaccaggactggctgaacggaaaggagtataagtgtaaggtgtccaacaagggactgccatccagcatcgagaagaccatctccaagaccaagggacagccaagagagccacaggtgtataccctgccaccatccagagaggagatgaccaagaaccaggtgtccctgacctgtctggtgaagggattctatccatccgacatcgccgtggagtgggagtccaacggacagccagagaacaactataagaccacccctccaatgctggactccgacggatccttcttcctgtattccaagctgaccgtggacaagtccagatggcagcagggaaacgtgttctcttgttccgtgatgcacgaggccctgcacaaccactatacccagaagagcctgtccctgtctccaggaaag